(19) United States
(12) Patent Application Publication
(10) Pub. No.: US 2024/0274424 A1
(43) Pub. Date: Aug. 15, 2024

(54) TANDEM MASS SPECTROMETER AND METHOD OF TANDEM MASS SPECTROMETRY

(71) Applicant: Thermo Fisher Scientific (Bremen) GmbH, Bremen (DE)

(72) Inventor: Hamish Stewart, Bremen (DE)

(73) Assignee: Thermo Fisher Scientific (Bremen) GmbH, Bremen (DE)

(21) Appl. No.: 18/433,245

(22) Filed: Feb. 5, 2024

(30) Foreign Application Priority Data
Feb. 6, 2023 (GB) 2301649.6

(51) Int. Cl. H01J 49/00; H01J 49/40; H01J 49/42

(52) U.S. Cl. CPC H01J 49/0081; H01J 49/0009; H01J 49/0036; H01J 49/005; H01J 49/40; H01J 49/425

(57) ABSTRACT

A method of tandem mass spectrometry for analysing precursor ions across a mass to charge (m/z) range of interest is provided. The method comprises analysing some of the precursor ions across the m/z range of interest in the MS1 domain using a first mass analyser of a tandem mass spectrometer operated at a first sensitivity. The method also comprises analysing some of the precursor ions across the m/z range of interest in the MS1 domain using a second mass analyser of the tandem mass spectrometer operated at a second sensitivity, wherein the second sensitivity is higher than the first sensitivity. The analysis in the MS1 domain performed by the second mass analyser is performed concurrently with the analysis performed in the MS1 domain by the first mass analyser. The method also comprises combining data from the MS1 analyses performed by the first and second mass analysers to identify and/or quantify precursor ions. The method also comprises analysing some of the precursor ions in the MS2 domain using the second mass analyser of the tandem mass spectrometer.

### CROSS REFERENCE TO RELATED APPLICATION

This application claims priority to GB application 2301649.6 filed Feb. 6, 2023, and that GB application is incorporated by reference herein.

### FIELD OF THE DISCLOSURE

The present disclosure relates to mass spectrometry. In particular, the present disclosure relates to a tandem mass spectrometer and a methods of tandem mass spectrometry.

### BACKGROUND

Mass spectrometry is a long-established technique for identification and quantitation of often complex mixtures of large organic molecules. Proteins in particular, comprising large numbers of amino acids, are typically of significant molecular weight. Thus, accurate identification and quantitation of the protein by direct mass spectrometric measurement is challenging. It is thus well known to carry out fragmentation of the precursor sample ions. A variety of fragmentation techniques are known, which may result in the generation of different fragment ions from the precursor ions. Moreover, the fragmentation mechanism may be affected by different applied fragmentation energies.

One known method of acquiring mass spectrometry data involving the fragmentation of precursor ions is known as data independent analysis/acquisition (DIA) mass spectrometry.

DIA seeks to determine what is present in a sample of potentially unknown identity. To determine the molecular structure of sample molecules, a mass spectrometer is first used to mass analyse all sample ions (precursor ions) within a mass to charge ratio (m/z) range of interest. Such a scan is often denoted as an MS1 scan. Selected sample ions are then fragmented, and the resulting fragments are subsequently mass analysed. The scan of the fragmented ions is often denoted as an MS2 scan.

An example of a DIA methodology is further described in U.S. Pat. No. 10,699,888 where a tandem mass spectrometer comprising an orbital trapping mass analyser and a Time Of Flight (TOF) mass analyser is used. The orbital trapping analyser is used to perform a series of relatively wide mass-to-charge (m/z) range MS1 scans, while the TOF performs a plurality of narrow m/z range MS2 scans.

Another method of acquiring mass spectrometry data involving the fragmentation of ions is known as data dependent analysis/acquisition (DDA) mass spectrometry. DDA mass spectrometry seeks to confirm that one or more species is/are present in a given sample. Methods of DDA identify a fixed number of precursor ion species and select and analyse those via mass spectrometry. The determination of which precursor ion species are of interest in DDA may be based upon intensity ranking (for example, the top ten most abundant species as observed by peaks in a MS1 spectrum), and/or by defining an “inclusion list” of precursor mass spectral peaks (for example by user selection), from which MS2 spectra are always acquired regardless of the intensity ranking of the peak in the MS1 mass spectrum. Furthermore, an “exclusion list” of peaks in MS1 can be defined, for example by a user, based e.g. on prior knowledge of the expected sample contents. A DDA methodology is further described in Hu A, Noble WS and Wolf-Yadlin A. “Technical advances in proteomics: new developments in data-independent acquisition” [version 1; peer review: 3 approved]. F1000Research 2016, 5(F1000 Faculty Rev):419.

Another data acquisition method for mass spectrometry termed “BoxCar”, or “HDR”, is disclosed in Meier et al, Nature Methods, 2018, 15, 440-448. This method involves splitting a broad mass range into many separate isolation windows. Each isolation window has a different fill time into a C-trap depending on the ion current of the isolation window. This workflow efficiently redistributes the C-Trap ion capacity between analytes, but also greatly increases the time required to accumulate ions, especially when a substantial number of windows is used. In particular, a relatively long fill time is required for low ion current isolation windows. Further, additional time is required to switch the quadrupole and ion source voltages between isolation windows. Thus, BoxCar/HDR data acquisition may quickly impact the rate at which MS2 scans may be made.

Methods of mass spectrometry involving the analysis of precursor ions (MS1 scans) and fragments ions (MS2 scans) may be performed by a tandem mass spectrometer. An example of a tandem mass spectrometer is disclosed in U.S. Pat. No. 10,699,888.

Against this background, the present disclosure seeks to provide an improved, or at least commercially useful alternative, method of tandem mass spectrometry.

### SUMMARY

According to a first aspect of the disclosure, a method of tandem mass spectrometry for analysing precursor ions across a mass to charge (m/z) range of interest is provided. The method comprises analysing some of the precursor ions across the m/z range of interest in the MS1 domain using a first mass analyser of a tandem mass spectrometer operated at a first sensitivity. The method also comprises analysing some of the precursor ions across the m/z range of interest in the MS1 domain using a second mass analyser of the tandem mass spectrometer operated at a second sensitivity, wherein the second sensitivity is higher than the first sensitivity. The analysis in the MS1 domain performed by the second mass analyser is performed concurrently with the analysis performed in the MS1 domain by the first mass analyser. The method also comprises combining data from the MS1 analyses performed by the first and second mass analysers to identify and/or quantify precursor ions. The method also comprises analysing some of the precursor ions in the MS2 domain using the second mass analyser of the tandem mass spectrometer.

According to the method of the first aspect, the tandem mass spectrometer is used to perform analyses in the MS1 domain using a first mass analyser and a second mass analyser. The first and second mass analysers are operated at different sensitivities. In particular, the second mass analyser is operated at a higher sensitivity than the first mass analyser in addition to being used to perform analysis of the precursor ions in the MS2 domain. As such, the MS1 analysis performed by the second mass analyser is used to supplement the data from the MS1 analysis performed by the first mass analyser. The higher sensitivity MS1 data obtained by the second mass analyser can in particular be used to identify lower intensity precursor ions that may not be identified by the MS1 analysis performed by the first mass analyser. That is to say, where the dynamic range of the first mass analyser may not reliably identify relatively low abundance precursor ions, the MS1 analysis performed by the second mass analyser at a higher sensitivity may identify said lower abundance precursor ions.

By sensitivity, it is understood that the sensitivity of the mass analyser reflects the intensity of the signal recorded by the mass analyser for a fixed quantity of a given ion. As such, analysing a quantity of a given ion at a relatively high sensitivity will generate mass peaks with a higher intensity than a mass analysis of the same quantity of a given ion mass analysed at a lower sensitivity. It will be appreciated that some mass analysers may be operated at different sensitivities. Furthermore, different types of mass analysers may be capable of operating at different ranges of sensitivities. For example, a TOF mass analyser may be capable of detecting single ions (i.e. operating at a relatively high intensity), while other mass analysers, such as an orbital trapping mass analyser may be capable of operation at a relatively lower range of sensitivities.

By performing the MS1 and MS2 analyses using the second mass analyser concurrently with the MS1 analysis performed by the first mass analyser, the method according to the first aspect may identify a wider range of precursor ions present in the sample in real time. For example, the MS1 data from the second mass analyser may be used to identify a wider range of precursor ions due to the higher sensitivity of the second mass analyser. This data may be combined with the MS1 data from the first mass analyser in order to improve the identification of precursor ions. For example, the combined data may be used to inform, or update the precursor ions to be analysed in the MS2 domain. For example, in some embodiments the MS1 analyses performed at different sensitivities may also have different mass accuracies, and/or different peak acceptances. By combining MS1 data from two different MS1 analyses performed by two mass analysers operating in parallel, the method of tandem mass spectrometry may provide improved identification of precursor ions, in particular low abundance precursor ions.

In some embodiments, the first mass analyser is operated at a first sensitivity and a first mass accuracy, and the second mass analyser analyses some of the precursor ions in the MS1 domain at a second sensitivity and a second mass accuracy, wherein the second mass accuracy is lower than the first mass accuracy. Accordingly, the first mass analyser, for example an orbital trapping mass analyser may be used to perform an MS1 analysis with a relatively high mass accuracy, but also a relatively low sensitivity. The second mass analyser (e.g. a TOF mass analyser) may perform an MS1 analysis in tandem at a higher sensitivity, but lower mass accuracy, than the first mass analyser. Advantageously, by performing a second MS1 analysis at a higher sensitivity, relatively low abundance precursor ion peaks may be detected. In addition, the first MS1 analysis provides relatively mass accurate information of the precursor ions detected. Thus, by combining data from the first and second mass analysers, precursor ions may be identified and/or quantified with across a large dynamic range and with improved mass accuracy.

In some embodiments, the data from the MS1 analysis performed by the first and second mass analysers may be combined to generate a combined list of precursor ion peaks in the MS1 domain. That is to say, the MS1 analysis performed by the first and second mass analysers at different sensitivities may each identify a different set of precursor ion peaks in the MS1 domain. In particular, the MS1 analysis performed by the second mass analyser at a higher sensitivity may identify precursor ion peaks corresponding to precursor ions having a relatively low intensity that may not have been detectable in the MS1 analysis performed by the first mass analyser. Thus, the combined list of precursor ion peaks may comprise more precursor ion peaks than would have otherwise been present in a list of precursor ion peaks based on the MS1 analysis performed by the first mass analyser. Further, the second mass analyser may not accurately record the peak shape of a precursor ion peak corresponding to a relatively high intensity precursor ion due to the high sensitivity of the second mass analyser. Thus, the data from the MS1 analysis performed by the first mass analyser may provide a more accurate representation of a relatively high intensity precursor ion peak due to the lower sensitivity of the first mass analyser. In particular, the combined list of precursor ion peaks in the MS1 domain may provide a more complete list of precursor ion peaks, and also a have a more accurate list of the mass to charge ratios of precursor ion peaks in the MS1 domain.

In some embodiments, the combined list of precursor ion peaks comprises a first set of precursor ion peaks identified from the MS1 analysis performed by the first mass analyser, and a second set of precursor ion peaks identified from the MS1 analysis performed by the second mass analyser. The combined list may be filtered to remove any precursor ion peaks which are repeated between the first and second sets of precursor ion peaks. Thus, the combined list of precursor ion peaks may be based on data from MS1 analyses performed at different sensitives, and subsequently filtered to take advantage of the advantages associated with each of the MS1 analysis.

In some embodiments, the second set of precursor ion peaks is thresholded to remove any precursor ion peaks below a first predetermined intensity level. In some embodiments, the second set of precursor ion peaks maybe thresholded to remove any precursor ion peaks above a second predetermined intensity level.

Thus, the second set of precursor ion peaks may be filtered to remove any low intensity peaks in the mass spectrum which may be indistinguishable from noise. The second set of precursor ion peaks may also be filtered to remove any precursor ion peaks having a relatively large intensity, which would be expected to have been recorded by the MS1 analysis performed by the first mass analyser. As such, the second set of precursor ion peaks may be thresholded to focus on the identification of precursor ion peaks associated with relatively low abundance precursor ions.

In some embodiments, a first precursor ion peak generated by the second mass analyser, and a corresponding first precursor ion peak generated by the first mass analyser are used to calibrate the tandem mass spectrometer. The calibration is used to identify any precursor ion peaks which are repeated between the first and second sets of precursor ion peaks. For example, the calibration may be used to determine an association between a mass to charge m/z ratio of a first precursor ion peak generated by the second mass analyser and a m/z ratio of the corresponding first precursor ion peak generated by the first mass analyser. The association may then be used to identify other precursor ion peaks which are repeated between the first and second sets of precursor ion peaks.

In some embodiments the analyses performed in the MS2 domain are based on the precursor ions identified by the MS1 analyses. For example, the combined list of precursor ion peaks may be used to generate a list of precursor ions which are to be further analysed in the MS2 domain. In particular, it will be appreciated that by identifying a wider range of precursor ions in the MS1 domain due to the use of two different mass analysers operating at different sensitivities, the precursor ions to be analysed in the MS2 domain may be more representative of the precursor ions present in the sample.

According to this disclosure, it will be appreciated that a reference to the analysis of the precursor ions in the MS2 domain is understood to mean that the precursor ions are fragmentated before being analysed by the second mass analyser. That is to say, analysing the precursor ions in the MS2 domain comprises fragmenting the precursor ions to generate product ions, and analysing the product ions using the second mass analyser.

In some embodiments, the method comprises performing a plurality of analysis cycles using the tandem mass spectrometer, wherein each cycle comprises: performing a single analysis across the m/z range of interest in the MS1 domain using the first mass analyser; and performing a single analysis across the m/z range of interest in the MS1 domain using the second mass analyser, and performing analyses of some of the precursor ions in the MS2 domain using the second mass analyser. The analyses performed in the MS1 and MS2 domains by the second mass analyser are performed concurrently with the single analysis in the MS1 domain performed by the first mass analyser. As such, the method according to the first aspect may be a methodology of tandem mass spectrometry wherein the analysis cycle is repeated over a period of time in order to analyse precursor ions. In particular, the methodology may be particularly applicable where the precursor ions to be analysed are varying over time for example due to the precursor ions being provided by an ion source coupled to a chromatography apparatus.

In some embodiments, the method comprises performing a plurality of analysis cycles using the tandem mass spectrometer wherein each analysis cycle comprises: performing a single analysis of the m/z range of interest in the MS1 domain using the first mass analyser, and subdividing the m/z range of interest into a plurality of m/z subranges and performing an analysis across each m/z subrange of interest in the MS1 domain using the second mass analyser, and performing analyses of some of the precursor ions in the MS2 domain using the second mass analyser. The analyses performed in the MS1 and MS2 domains by the second mass analyser are performed concurrently with the single analysis in the MS1 domain performed by the first mass analyser.

According to some embodiments, an analysis cycle may include performing a plurality of MS1 analyses of m/z subranges of interest using the second mass analyser. By segmenting the mass range of interest into subranges and analysing them using the second mass analyser at a higher sensitivity the second mass analyser may identify precursor ions having a relatively low abundance. In particular, by subdividing the mass range of interest, and analysing each subrange separately, the ion current used to populate each m/z subrange may be adjusted. That is to say, subdividing the m/z range of interest into a plurality of m/z subranges may improve the identification of relatively low abundance precursor ions using the second mass analyser.

In some embodiments, an analysis cycle may comprise performing a single analysis across of the m/z range of interest in the MS1 domain using the first mass analyser. The analysis cycle may also comprise performing a plurality of analyses across the m/z range of interest in the MS1 domain using the second mass analyser.

In some embodiments, for each analysis cycle the data from the MS1 analyses performed by the second mass analyser may be averaged. As such, an averaged MS1 mass spectrum may be generated using the second mass analyser. As the second mass analyser is operated with a higher sensitivity than the first mass analyser, averaging the MS1 mass spectra generated by the second mass analyser may increase the number of precursor ion peaks which are detectable in the MS1 data. In particular, relatively low abundance precursor ions (e.g. around 1 to 3 precursor ions present per MS1 analysis) may be more reliably detected across a plurality of MS1 analyses. In some embodiments, at least 5 MS1 analyses, more preferably at least 10 MS1 analyses, may be performed by the second mass analyser and averaged.

In some embodiments, averaging the data from the MS1 analyses further comprises thresholding the averaged data to remove precursor ion peaks below a third intensity level. For example, in some embodiments, the step of averaging the MS1 analyses may result in a number of low intensity precursor ion peaks (e.g. corresponding to single ion intensity) which may be the result of ion scattering during mass analysis. Such precursor ion peaks are not “true” peaks representative of a precursor ion, and the accuracy of the averaged data may be improved by thresholding the averaged data to remove the “false” precursor ion peaks.

It will be appreciated that the MS1 analyses performed by the second mass analyser may be performed at any time during an analysis cycle. For example, in some embodiments, the MS1 analyses performed by the second mass analyser may be performed towards the start of each MS1 analysis performed by the first mass analyser. For example, the MS1 analyses performed by the second mass analyser may be performed during an initial period of the MS1 analysis performed by the first mass analyser. In some embodiments, the initial period may be no greater than: 30%, or 20%, of the total (expected) duration of the MS1 analysis performed by the first mass analyser. As such, it will be appreciated that the initial period extends from the start of the MS1 analysis performed by the first mass analyser to the end of the initial period (i.e. when 30% of the total (expected) duration of the MS1 analysis has passed).

In some embodiments, it may be advantageous to distribute the MS1 analyses performed by the second mass analyser throughout the duration of the of the MS1 analysis performed by the first mass analyser. As such, in some embodiments where a plurality of MS1 analyses are performed by the second mass analyser, the MS1 analyses may be distributed evenly across the duration of the MS1 analysis performed by the first mass analyser. That is to say, in some embodiments the plurality of MS1 analyses to be performed by the second mass analyser may be interleaved with the MS2 analyses to be performed by the second mass analyser. For example, for each analysis cycle, at least 3, 5, or 7 analyses may be performed in the MS1 domain by the second mass analyser. In some embodiments, the analyses performed in the MS1 domain are interleaved evenly throughout the duration of the analysis performed in the MS1 domain by the first mass analyser. By interleaving the MS1 analyses in this manner, the MS1 data obtained by the second mass analyser may more accurately characterise a change in the precursor ions being analysed over time (for example due to the elution of a chromatographic peak from a chromatographic separation apparatus).

In some embodiments, each analysis cycle further comprises performing a gain control analysis using the first mass analyser and/or the second mass analyser. An injection time for each of the MS1 analyses performed by the first and/or second mass analysers may be adjusted based on the gain control analyses. As such, the tandem mass spectrometer may be adjusted based on the gain control analysis in order to control the ion current used to populate the precursor ions used for each of the MS1 analyses. This may be particularly applicable to the MS1 analyses performed by the second mass analyser in order to improve the identification of relatively low abundance precursor ions.

In some embodiments, the precursor ions to be analysed are filtered to remove most or all of any singly-charged precursor ions. For example, the precursor ions may be filtered by a Field Asymmetric Ion Mobility Spectrometer (FAIMS). By filtering out the singly-charged precursor ions, mostly or only multiply charge precursor ions may be analysed by the methods of the first aspect. Multiply charged ions are more informative species to be analysed using MS2 analysis, thereby improving the efficiency of the methodology.

In some embodiments, the precursor ions to be analysed are provided by an ion source which is configured to ionise molecules provided from a chromatographic separation apparatus. For example, a sample may be provided to a chromatographic separation apparatus, wherein the sample elutes from the chromatographic separation apparatus. The eluting sample is then ionised by the ion source to generate a stream of precursor ions which are provided to the tandem mass spectrometer for analysis. It will be appreciated that sample molecules eluting from a chromatographic separation apparatus may elute at different time periods depending on the sample molecules present in the sample. Each different sample molecule may elute with its own chromatographic peak over a period of time. For example, some chromatographic peaks may have a duration of about 1 to 20 seconds depending on the chromatographic separation apparatus and the sample molecule. As such, the method of tandem mass spectrometry of the first aspect in some embodiments may be performed over the duration of a chromatographic peak. Preferably, the method of tandem mass spectrometry may be repeated a plurality of times over the duration of the chromatographic peak, such that the MS1 analyses performed by the first and second mass analysers are repeated over the duration of the chromatographic peak. For example, the MS1 analysis performed by the first mass analyser may preferably be performed at least 2, 3, 5, or 7 times over the duration of the chromatographic peak. Of course, it will be appreciated that for some particularly fast eluting chromatographic peaks, there may not be sufficient time to perform multiple repetitions of an analysis cycle according to the first aspect.

In some embodiments, data from the MS1 analyses performed by the first and second mass analysers may be used to identify a chromatographic peak eluting from the chromatographic separation apparatus. In some embodiments, the analyses to be performed by in the MS2 domain by the second mass analyser may be selected based on the identified chromatographic peak eluting from the mass spectrometer. Thus, the method of the first aspect may more accurately characterise a chromatographic peak eluting from the chromatographic separation apparatus.

In some embodiments, the first mass analyser is a mass analyser selected from the group comprising: an orbital trapping mass analyser, a Fourier-Transform Ion Cyclotron Resonance (FTICR) mass analyser, and a Time Of Flight (TOF) mass analyser. In some embodiments, the second mass analyser is a TOF mass analyser. As such, in some embodiments the first mass analyser and the second mass analyser may be different mass analysers. Alternatively, in some embodiments the first mass analyser and the second mass analyser may be the same type of mass analyser. In both cases, each mass analyser may be operable at different sensitivity ranges. Methods according to the first aspect operate the two mass analysers at different sensitivities in order to obtain information about relatively high abundance precursor ions and also information about relatively low abundance precursor ions. That is to say, methods according to the first aspect seek to acquire information in the MS1 domain about the precursor ions present over a larger dynamic range than would be possible using a single mass analyser. For example, in some embodiments, the second mass analyser may have a larger dynamic range than the first mass analyser.

In some embodiments, the first mass analyser is operated at a dynamic range to perform the respective MS1 analysis, and the second mass analyser is operated at a second dynamic range larger than the first dynamic range to perform the respective MS1 analysis. That is to say, in addition to the difference in sensitivities, the methods according to the first aspect may also operate the mass analysers at different dynamic ranges in order to obtain a more complete picture about the precursor ions in the MS1 domain.

According to a second aspect of the disclosure, a tandem mass spectrometer for analysing precursor ions across a mass to charge (m/z) range of interest is provided. The tandem mass spectrometer comprises a first mass analyser, a second mass analyser and a controller. The first mass analyser is configured to analyse precursor ions in the MS1 domain. The second mass analyser is configured to analyser precursor ions in the MS1 domain and the MS2 domain. The controller is configured to:
cause the first mass analyser to analyse some of the precursor ions across the m/z range of interest in the MS1 domain at a first sensitivity;
cause the second mass analyser to analyse some of the precursor ions across the m/z range of interest in the MS1 domain at a second sensitivity, wherein the second sensitivity is higher than the first sensitivity,
wherein the second mass analyser analyses the precursor ions in the MS1 domain concurrently with the first mass analyser analysing the precursor ions in the MS1 domain;
combine data from the MS1 analyses performed by the first and second mass analysers to identify and/or quantify precursor ions; and
cause the second mass analyser to analyse some of the precursor ions in the MS2 domain.

As such, it will be appreciated that the tandem mass spectrometer of the first aspect is configured to perform the method of the first aspect. In particular, it will be appreciated that the tandem mass spectrometer may include features configured to perform any of the optional features of the first aspect.

In particular, in some embodiments it will be appreciated that the tandem mass spectrometer may comprise a fragmentation device configured to fragment the precursor ions to generate product ions, wherein the product ions may be analysed to provide MS2 data.

In some embodiments, the tandem mass spectrometer may be configured to receive precursor ions from an ion source. The precursor ions to be analysed may be provided by the ion source which is configured to ionise molecules provided from a chromatographic separation apparatus. For example, a sample may be provided to a chromatographic separation apparatus, wherein the sample elutes from the chromatographic separation apparatus. The eluting sample is then ionised by the ion source to generate a stream of precursor ions which are provided to the tandem mass spectrometer for analysis.

According to a third aspect of the disclosure, a computer program comprising instructions to cause the tandem mass spectrometer of the second aspect to execute the steps of the method according to the first aspect is provided.

In accordance with a fourth aspect of the disclosure, a computer-readable medium having stored thereon the computer program of the third aspect is provided.

### BRIEF DESCRIPTION OF THE FIGURES

Embodiments of the disclosure will now be described with reference to the following non-limiting figures in which:

FIG. 1 shows a schematic diagram of a tandem mass spectrometer according to the present disclosure;

FIG. 2 shows a block diagram of a method of tandem mass spectrometry according to an embodiment of the disclosure;

FIG. 3 shows a further flow chart of a method of tandem mass spectrometry according to an embodiment of the disclosure;

FIGS. 4a and 4b show MS1 mass spectra of a calibration solution obtained by an orbital trapping mass analyser operating at a resolution of 120,000;

FIGS. 5a and 5b show MS1 mass spectra of a calibration solution obtained by an orbital trapping mass analyser operating at a resolution of 240,000;

FIGS. 6a and 6b show MS1 mass spectra of a calibration solution obtained by a time of flight mass analyser (single shot);

FIGS. 7a and 7b show an averaged MS1 mass spectra of a calibration solution obtained by from ten MS1 mass analyses performed by a time of flight mass analyser;

FIG. 8 shows a bar chart indicating the number of different precursor ion peaks identified using MS1 analyses of a calibration solution for different mass analyser and mass analysis techniques;

FIG. 9 shows a graph indicating the number of precursor ion peaks detected using a TOF mass analyser as a first intensity level (threshold) is varied; and

FIG. 10 shows a block diagram of a method of tandem mass spectrometry according to a further embodiment of the disclosure.

### DETAILED DESCRIPTION

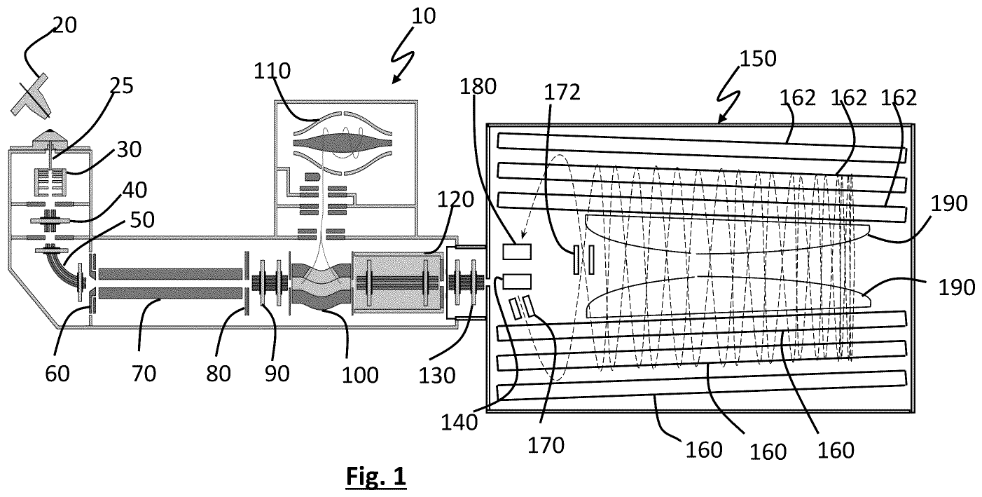

FIG. 1 shows a schematic arrangement of a tandem mass spectrometer 10 suitable for carrying out methods in accordance with embodiments of the present invention.

In FIG. 1, a sample to be analysed is supplied (for example from an autosampler) to a chromatographic apparatus such as a liquid chromatography (LC) column (not shown in FIG. 1). One such example of an LC column is the Thermo Fisher Scientific, Inc ProSwift® monolithic column which offers high performance liquid chromatography (HPLC) through the forcing of the sample carried in a mobile phase under high pressure through a stationary phase of irregularly or spherically shaped particles constituting the stationary phase. In the HPLC column, sample molecules elute at different rates according to their degree of interaction with the stationary phase.

The sample molecules thus separated via liquid chromatography are then ionized using an ion source to generate precursor ions. In the embodiment of FIG. 1, the ion source is an electrospray ionization source (ESI source) 20 which is at atmospheric pressure.

Precursor ions generated by the ESI source 20 then enter a vacuum chamber of the tandem mass spectrometer 10 and are directed by a capillary 25 into an RF-only S lens 30. The precursor ions are focused by the S lens 30 into an injection flatapole 40 which injects the precursor ions into a bent flatapole 50 with an axial field. The bent flatapole 50 guides (charged) precursor ions along a curved path through it whilst unwanted neutral molecules such as entrained solvent molecules are not guided along the curved path and are lost.

An ion gate (TK lens) 60 is located at the distal end of the bent flatapole 50 and controls the passage of the precursor ions from the bent flatapole 50 into a downstream mass selector in the form of a quadrupole mass filter 70. Alternatively, the S lens 30 may be operated as an ion gate and the ion gate (TK lens) 60 may be a static lens. The quadrupole mass filter 70 is typically but not necessarily segmented and serves as a band pass filter, allowing passage of a selected mass number or limited mass range whilst excluding precursor ions of other mass to charge ratios (m/z). The mass filter can also be operated in an RF-only mode in which it is not mass selective, i.e. it transmits substantially all m/z precursor ions. For example, the quadrupole mass filter 70 may be controlled by a controller (not shown in FIG. 1) to select a range of mass to charge ratios to pass of the precursor ions which are allowed to pass, whilst the other ions in the precursor ion stream are filtered out (not allowed to pass). As such, the mass filter 70 may filter the precursor ions based on the m/z range of interest.

Although a quadrupole mass filter 70 is shown in FIG. 1, the skilled person will appreciate that other types of mass selection devices may also be suitable for selecting precursor ions within the mass range of interest. For example, an ion separator as described in US-A-20150287585, an ion trap as described in WO-A-2013076307, an ion mobility separator as described in US-A-2012256083, an ion gate mass selection device as described in WO-A-2012175517, or a charged particle trap as described in U.S. Pat. No. 7,999,223, the contents of which are hereby incorporated by reference in their entirety. The skilled person will appreciate that other methods of selecting precursor ions according to ion mobility, differential mobility and/or transverse modulation may also be suitable.

The isolation of a plurality of precursor ions of different masses or mass ranges may also be performed using the method known as synchronous precursor scanning (SPS) in an ion trap. Furthermore, in some embodiments, more than one ion selection or mass selection device may be provided. For example, a further mass selection device may be provided downstream of the fragmentation chamber 120. In this way, MS³ or MSⁿ scans can be performed if desired (typically using the TOF mass analyser for mass analysis).

The tandem mass spectrometer 10 may be operated in various modes of operation in order to perform analysis of the precursor ions in the MS1 domain and/or the MS2 domain. In a first mode of operation, the precursor ions may be analysed in the MS1 domain using a first mass analyser (orbital trapping mass analyser 110).

In the first mode of operation, precursor ions may pass through a quadrupole exit lens/split lens arrangement 80 and into a first transfer multipole 90. The first transfer multipole 90 guides the mass filtered ions from the quadrupole mass filter 70 into a curved linear ion trap (C-trap) 100. The C-trap (first ion trap) 100 has longitudinally extending, curved electrodes which are supplied with RF voltages and end caps that to which DC voltages are supplied. The result is a potential well that extends along the curved longitudinal axis of the C-trap 100. In a first mode of operation, the DC end cap voltages are set on the C-trap so that ions arriving from the first transfer multipole 90 are captured in the potential well of the C-trap 100, where they are cooled. Cooled precursor ions reside in a cloud towards the bottom of the potential well of the C-trap 100. The injection time (IT) of the ions into the C-trap determines the number of precursor ions (ion population) that is subsequently ejected from the C-trap 100. From the C-trap 100, precursor ions may be directed to different parts of the tandem mass spectrometer 10, depending on the analysis to be performed.

Where precursor ions are to be analysed by the orbital trapping mass analyser 110 (first mass analyser), the precursor ions are ejected orthogonally from the C-trap towards the orbital trapping mass analyser 110. As shown in FIG. 1, the orbital trapping mass analyser 110 may be an Orbitrap® mass analyser sold by Thermo Fisher Scientific, Inc. The orbital trapping mass analyser 110 has an off-centre injection aperture and the precursor ions are injected into the orbital trapping mass analyser 110 as coherent packets, through the off-centre injection aperture. Precursor ions are then trapped within the orbital trapping mass analyser 110 by a hyperlogarithmic electric field and undergo back and forth motion in a longitudinal direction whilst orbiting around the inner electrode.

The axial (z) component of the movement of the ion packets in the orbital trapping mass analyser 110 is (more or less) defined as simple harmonic motion, with the angular frequency in the z direction being related to the square root of the mass to charge ratio of a given ion species. Thus, over time, precursor ions separate in accordance with their mass to charge ratio.

Precursor ions in the orbital trapping mass analyser 110 are detected by use of an image current detector (not shown) which produces a “transient” in the time domain containing information on all of the ion species as they pass the image current detector. The transient is then subjected to a Fast Fourier Transform (FFT) resulting in a series of peaks in the frequency domain. From these peaks, a mass spectrum, representing abundance/ion intensity versus m/z, can be produced.

In the configuration described above, the precursor ions within the mass range of interest (selected by the quadrupole mass filter 70) are analysed by the orbital trapping mass analyser 110 without fragmentation. The resulting mass spectrum is denoted MS1.

Although an orbital trapping mass analyser 110 is shown in FIG. 1, it will be appreciated that other mass analysers may be employed as the first mass analyser according to embodiments of this disclosure. For example, a Fourier Transform Ion Cyclotron Resonance (FTICR) mass analyser may be utilised as first mass analyser to analyse the precursor ions in the MS1 domain. Mass analysers, such as the orbital trapping mass analyser and Ion Cyclotron Resonance mass analyser, may also be used in the invention even where other types of signal processing than Fourier transformation are used to obtain mass spectral information from the transient signal (see for example WO 2013/171313, Thermo Fisher Scientific). In some embodiments, a TOF mass analyser may also be used in place of the orbital trapping mass analyser 110 to analyse the precursor ions in the MS1 domain.

In a second mode of operation of the tandem mass spectrometer 10, precursor ions may be analysed by the TOF mass analyser 150 (second mass analyser) in the MS1 domain. The precursor ions to be analysed by the second mass analyser may be mass filtered by the quadrupole mass filter 70. As such, the precursor ions may be filtered to include precursor ions from the m/z range of interest, or from a m/z subrange of interest.

In order for the TOF mass analyser 150 to analyse precursor ions, precursor ions may pass from the quadrupole exit lens/split lens arrangement 80 and first transfer multipole 90 into the C-trap 100 and continue their path through the C-trap 100 and into the fragmentation chamber 120. As such, the C-trap 100 may effectively be operated as an ion guide in the second mode of operation. Alternatively, cooled ions in the C-trap 100 may be ejected from the C-trap in an axial direction into the fragmentation chamber 120. As the precursor ions are to be analysed in the MS1 domain, the fragmentation chamber 120 is not used to fragment the precursor ions. Thus, the precursor ions may continue through the fragmentation chamber 120 and be ejected from the fragmentation chamber 120 at the opposing axial end to the C-trap 100. As such, the fragmentation chamber 120 may also effectively be operated as an ion guide in the second mode of operation.

The ejected precursor ions pass into a second transfer multipole 130. The second transfer multipole 130 guides the precursor ions from the fragmentation chamber 120 into an extraction trap (second ion trap) 140. The extraction trap 140 is a radio frequency voltage-controlled trap containing a buffer gas. For example, a suitable buffer gas is nitrogen at a pressure in the range 5×10⁴ mBar to 1×10⁻² mBar. The extraction trap has the ability to quickly switch off the applied RF voltage and apply a DC voltage to extract the trapped precursor ions. A suitable flat plate extraction trap, also referred to as a rectilinear ion trap, is further described in U.S. Pat. No. 9,548,195 (B2). Alternatively, a second C-trap may also be suitable for use as a second ion trap.

The extraction trap 140 is provided to form an ion packet of precursor ions, prior to injection into the TOF mass analyser 150. The extraction trap 140 accumulates fragmented ions prior to injection of the precursor ions into the TOF mass analyser 150.

Although an extraction trap 140 (ion trap) is shown in the embodiment of FIG. 1, the skilled person will appreciate that other methods of forming an ion packet of precursor ions will be equally suitable for the present invention. For example, relatively slow transfer of ions through a multipole can be used to affect bunching of ions, which can subsequently be ejected as a single packet to the TOF mass analyser. Alternatively, orthogonal displacement of precursor ions may be used to form a packet. Further details of these alternatives are found in US-A-20030001088 which describes a travelling wave ion bunching method, the contents of which are herein incorporated by reference.

In FIG. 1, the TOF mass analyser 150 shown is a multiple reflection time of flight mass analyser (mr-TOF). The TOF mass analyser 150 is constructed around two opposing ion mirrors 160, 162, elongated in a drift direction. The mirrors are opposed in a direction that is orthogonal to the drift direction. The extraction trap 140 injects ions into the first mirror 160 and the ions then oscillate between the two mirrors 160, 162. The angle of ejection of ions from the extraction trap 140 and additional deflectors 170, 172 allow control of the energy of the ions in the drift direction, such that ions are directed down the length of the mirrors 160, 162 as they oscillate, producing a zig-zag trajectory. The mirrors 160, 162 themselves are tilted relative to one another, producing a potential gradient that retards the ions' drift velocity and causes them to be reflected back in the drift dimension and focused onto a detector 180. The tilting of the opposing mirrors would normally have the negative side-effect of changing the time period of ion oscillations as they travel down the drift dimension. This is corrected with a stripe electrode 190 (to act as a compensation electrode) that alters the flight potential for a portion of the inter-mirror space, varying down the length of the opposing mirrors 160, 162. The combination of the varying width of the stripe electrode 190 and variation of the distance between the mirrors 160, 162 allows the reflection and spatial focusing of ions onto the detector 180 as well as maintaining a good time focus. A mr-TOF suitable for use in the present invention is further described in US-A-2015028197, the contents of which are hereby incorporated by reference in its entirety.

Precursor ions accumulated in the extraction trap 140 are injected into the TOF mass analyser 150 (second mass analyser) as a packet of ions. The time taken for the precursor ions to arrive at the detector 180 is recorded by the TOF mass analyser 150 and used to generate an MS1 mass spectrum of the precursor ions.

The MS1 analysis performed by the TOF mass analyser 150 is performed at a second sensitivity. According to embodiments of this disclosure, the TOF mass analyser performs the MS1 analysis at a second sensitivity which is greater than the first sensitivity of the MS1 analysis performed by the orbital trapping mass analyser 110. For example, the TOF mass analyser may be configured to operate at single ion sensitivity. That is to say, the TOF mass analyser 150 may be configured to generate a signal associated with the arrival of a single ion at the detector of the TOF mass analyser 150 which has sufficient intensity to be distinguishable from the background noise.

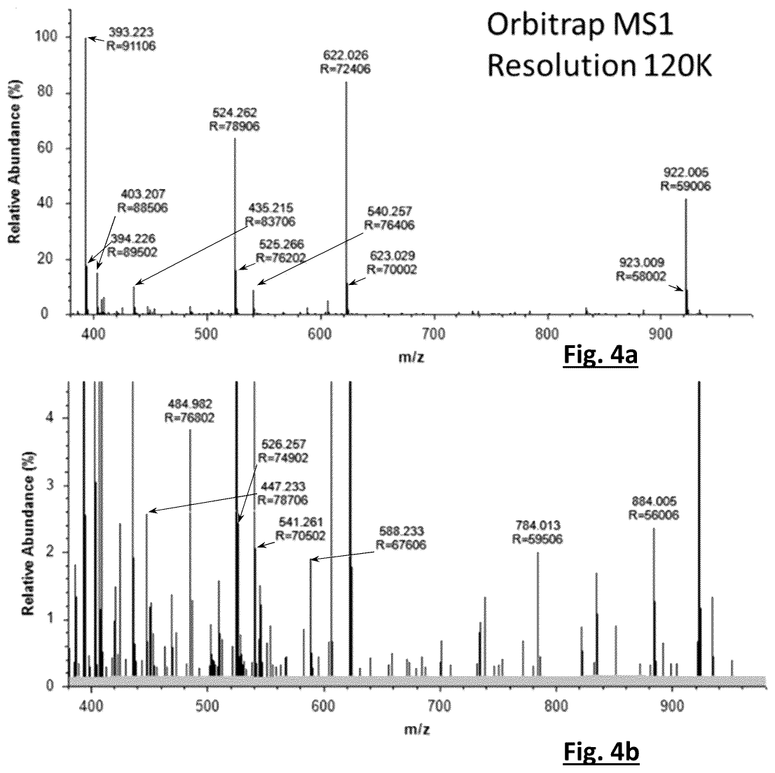

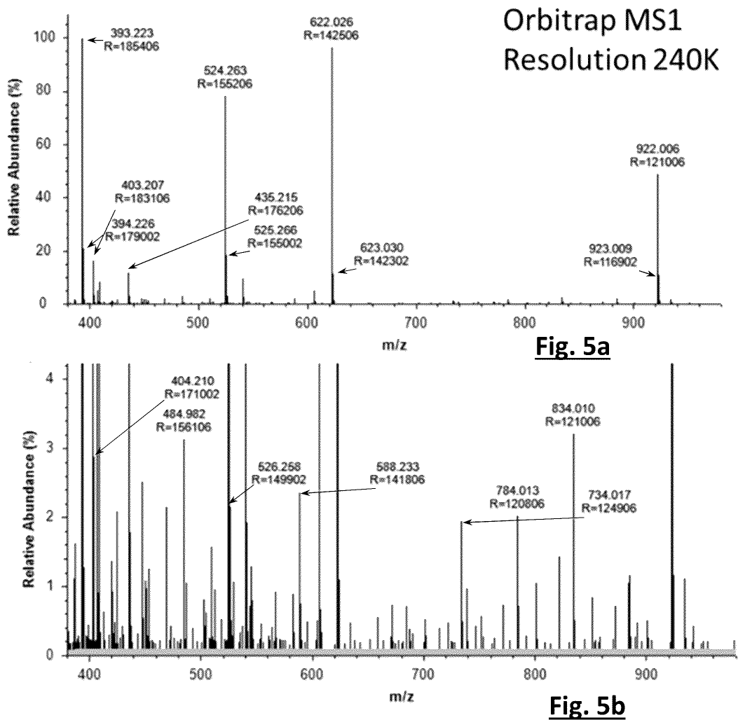

The skilled person will be aware of the sensitivity of different mass analysers and the effect of different operating conditions on the sensitivity of a given mass analyser. For example, according to this disclosure, one measure of sensitivity of a mass analyser may be the minimum number of ions required in an analysis to generate a peak which is distinguishable from background noise. As noted above, the TOF mass analyser 150 may be operated at single ion sensitivity (i.e. a single ion of a given mass may generate a corresponding peak). Other mass analysers (e.g. orbital trapping mass analyser 110) may require a plurality of ions to be present in order to generate a detectable peak (e.g. see discussion of FIGS. 4a, 5b, 5a and 5b below).

It will be appreciated that the TOF mass analyser also performs the MS1 analysis with an associated second (mass) accuracy.

In a third mode of operation of the tandem mass spectrometer 10, the TOF mass analyser 150 (second mass analyser) may be used to analyse the precursor ions in the MS2 domain.

In order to analyse the precursor ions in the MS2 domain, some of the precursor ions may be transferred from the quadrupole mass filter 70 to the fragmentation chamber 120 in a manner similar to second mode of operation discussed above. The precursor ions to be transferred may be mass selected by the quadrupole mass filter 70 to include targeted precursor ion species, or a m/z subrange of interest, as discussed in more detail below.

The fragmentation chamber 120 is, in the tandem mass spectrometer 10 of FIG. 1, a higher energy collisional dissociation (HCD) device. A collision gas may be supplied to the fragmentation chamber 120. When precursor ions are to be fragmented, the kinetic energy of the precursor ions may be increased, so that precursor ions arriving at the fragmentation chamber 120 and colliding with collision gas molecules results in fragmentation of the precursor ions into fragment ions.

Although an HCD fragmentation chamber 120 is shown in FIG. 1, other fragmentation devices may be employed instead, employing such methods as collision induced dissociation (CID), electron capture dissociation (ECD), electron transfer dissociation (ETD), photodissociation, and so forth.

Fragmented ions may be ejected from the fragmentation chamber 120 at the opposing axial end to the C-trap 100. The ejected fragmented ions pass into the second transfer multipole 130 and into the extraction trap 140 where they are accumulated. The fragmented ions may then be injected into the TOF mass analyser 150 as described above.

It will be appreciated that in some embodiments, the first mass analyser (orbital trapping mass analyser 110) and the second mass analyser (TOF mass analyser 150) may be operated concurrently. That is to say, it will be appreciated that the tandem mass spectrometer 10 may be operated in a first mode of operation concurrently with the second or third mode of operation.

The tandem mass spectrometer 10 may be under the control of a controller which, for example, is configured to control the timing of ejection of the trapping components, to set the appropriate potentials on the electrodes of the quadrupole etc. so as to focus and filter the ions, to capture the mass spectral data from the orbital trapping device 110, to capture the mass spectral data from the orbital trapping mass analyser 110 and the TOF mass analyser 150, control the sequence of MS1 and MS2 scans and so forth. It will be appreciated that the controller may comprise a computer that may be operated according to a computer program comprising instructions to cause the tandem mass spectrometer to execute the steps of the method according to the present invention.

It is to be understood that the specific arrangement of components shown in FIG. 1 is not essential to the methods described herein. Indeed other arrangements for carrying out the methods of embodiments of the present invention are suitable.

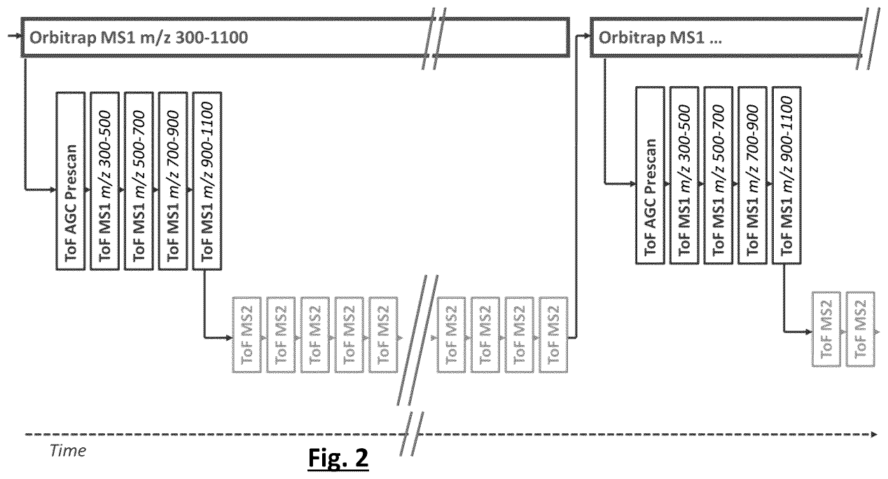

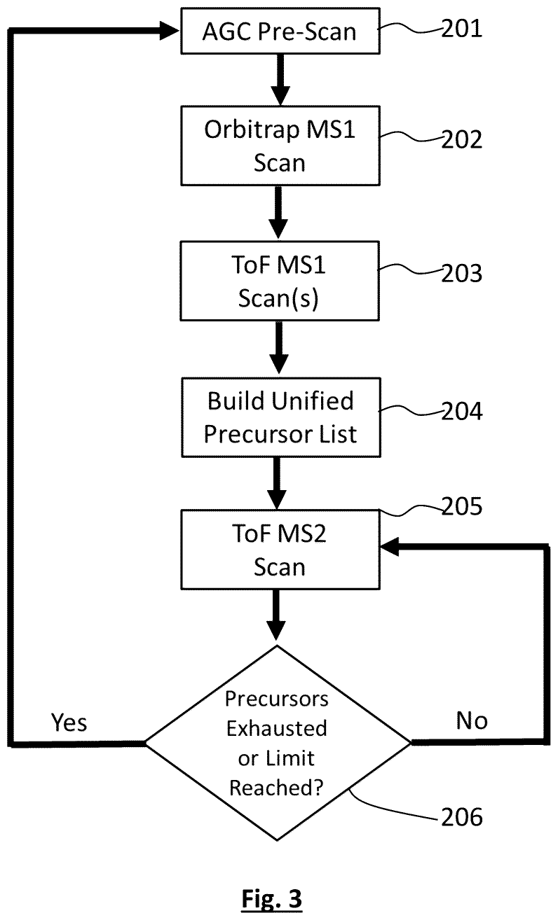

An embodiment of the method will now be described with reference to FIGS. 2 and 3, in which sample molecules are supplied from a liquid chromatography (LC) column as part of the exemplary apparatus described above (as shown in FIG. 1).

In the embodiment of the invention, the sample molecules are supplied from the LC column. As such, the methodology according to the present invention may acquire data about the sample over a duration corresponding to a duration of one or more chromatographic peaks of the sample supplied from the LC column. As such, the controller may be configured to perform an analysis cycle in accordance with an embodiment of this disclosure over the duration of a chromatographic peak. In some embodiments, the analysis cycle according to embodiments of this disclosure may be repeated a plurality of time as the sample elutes from the LC column.

As shown in FIG. 2, an orbital trapping mass analyser 110 (denoted “Orbitrap”) is utilised to perform a plurality of MS1 scans across a m/z range of interest. For example, as shown in FIG. 2, the m/z range of interest to be analysed is 300-1100 m/z. As shown in FIG. 2, for each analysis cycle, the orbital trapping mass analyser 110 is used to perform a single MS1 analysis of precursor ions within the m/z range of interest (300-1100 m/z). The precursor ions to be analysed may be provided via the quadrupole mass filter 70, wherein the precursor ions are filtered based on the m/z range of interest.

As shown in FIG. 2, the MS1 analysis performed by the orbital trapping analyser may be performed over an extended period of time (relative to the duration of the MS1 and MS2 analyses performed by the second mass analyser). Accordingly, the MS1 analysis performed by the orbital trapping mass analyser may have a relatively high mass accuracy and/or resolution relative to the MS1 analyses performed by the TOF mass analyser 150. In some embodiments, the MS1 analysis performed by the orbital trapping analyser may have a resolution of at least: 80,000, 100,000, 120,000, 150,000, 200,000, or 240,000. For example, in the embodiment of FIG. 2, the MS1 analysis performed by the orbital trapping mass analyser may have a duration of at least 250 ms, wherein the resolution of the resulting MS1 spectrum is about 120,000. In other embodiments, the MS1 analysis performed by the orbital trapping mass analyser 150 may have a duration of about 512 ms and a resolution of 240,000.

In general, increasing the resolution at which the MS1 analysis is performed decreases the noise baseline for the analysis. For example, when the orbital trapping mass analyser 110 of FIG. 1 is operated at a resolution of about 120,000 the noise baseline is believed to be equivalent to about 3.8 ions. When operated at a resolution of about 240,000, the noise baseline is believed to be about 2.6 ions. Thus, even when the orbital trapping mass analyser is operated at relatively high resolutions, it will be appreciated that several ions of any single analyte must be present in order to be detected by the orbital trapping mass analyser 110.

By using an orbital trapping mass analyser 110 (or any other relatively accurate mass analyser), the MS1 analysis using the first mass analyser is performed with a high degree of mass accuracy. Preferably, the MS1 analysis is performed with a mass accuracy of less than 5, or more preferably less than 3 parts per million (ppm). Parts per million mass accuracy Δm of a mass analyser may be determined as the difference between the measured mass of an ion mᵢ and the actual mass of an ion mₐ, divided by the actual mass of the ion, multiplied by 10⁶, as shown below:

$$Δ m = \frac{( {m}_{i} - {m}_{a} )}{{m}_{a}} \times {10}^{6}$$

Examples of mass spectra obtained by the orbital trapping mass analyser 110 are shown in FIGS. 4a, 4b, 5a, and 5b. In each case, the sample analysed is Pierce® FlexMix® Calibration Solution produced by Thermo Fisher Scientific®. Said calibration solution is a mixture of a mixture of 16 highly pure, ionizable components having a mass range from 50 m/z to 3000 m/z, and designed for both positive and negative ionization calibrations. FIGS. 4a and 4b show mass spectra obtained by the orbital trapping mass analyser 110 of FIG. 1 when performing an MS1 analysis of the sample (calibration solution) at a resolution of 120,000. FIG. 4a shows the relatively high abundance precursor ion peaks identified in the analysis, while FIG. 4b shows a magnified view of the precursor ion peaks of FIG. 4a having a relative abundance of less than 5%.

FIGS. 5a and 5b show mass spectra obtained by the orbital trapping mass analyser 110 of FIG. 1 when performing an MS1 analysis of the sample (calibration solution) at a resolution of 240,000. FIG. 5a shows the relatively high abundance precursor ion peaks identified in the analysis, while FIG. 5b shows a magnified view of the precursor ion peaks of FIG. 5a having a relative abundance of less than 5%.

It will be appreciated from FIGS. 4a, 4b, 5a, 5b, that by increasing the resolution of the orbital trapping mass analyser 110, a greater number of precursor ion peaks may be detected in the MS1 domain. For example, as observed in the experiment of FIGS. 4a and 4b, the orbital trapping mass analyser 110 when operated at a resolution of 120,000 had a sensitivity sufficient to distinguish a precursor ion peak generated by about 3.8 precursor ions from baseline noise. At a resolution of 240,000 (FIGS. 5a and 5b), the orbital trapping mass analyser has a sensitivity to distinguish precursor ion peaks corresponding to about 2.6 ions from baseline noise. As such, it will be appreciated that even when the orbital trapping mass analyser 110 is operated at relatively high resolution and relatively high sensitivity, several ions of any single analyte must still be present in order to be reliably detected by the orbital trapping mass analyser 110. That is to say, it is not generally practical/possible to detect precursor ion peaks associated with a single precursor ion (single ion sensitivity) using orbital trapping mass analyser 110. To the extent that it is possible to achieve single ion sensitivity, such an MS1 analysis using orbital trapping mass analyser 110 would likely require significant transient times, or other time-consuming ion processing steps, such that the resulting MS1 analysis would not be easily compatible with a normal mass spectrometry workflow. For example, it is preferable that multiple mass analyses can be performed on a timescale compatible with a chromatographic peak eluting from a chromatography system.

In tandem with the MS1 analysis performed by the orbital trapping mass analyser 110, one or more MS1 scans are performed by the second mass analyser (TOF mass analyser 150), for example as shown in FIG. 2. A plurality of MS2 scans are also performed by the TOF mass analyser 150. As shown in FIG. 2, the MS1 and MS2 analyses performed by the TOF mass analyser 150 are performed concurrently with the MS1 analysis performed by the orbital trapping mass analyser 110.

In order to perform an MS1 scan with the TOF mass analyser 150, precursor ions are transported from the ion source 20 to the extraction trap 140 as described above.

In the embodiment of FIG. 2, a plurality of MS1 analyses are performed by the TOF mass analyser 150, wherein each MS1 analysis is performed on a different m/z subrange within the mass range of interest (300-1100 m/z). As such, the mass range of interest is segmented into a plurality of m/z subranges. In the embodiment of FIG. 2, each m/z subrange has a m/z range of 200 m/z.

In order to perform a single MS1 analysis of a mass range segment, sample molecules (e.g. molecules of sample solution) from an LC column are ionised and injected into the tandem mass spectrometer 10 in a similar manner to the process for performing an MS1 analysis using the orbital trapping mass analyser 110.

Once the precursor ions for the MS1 analysis reach the quadrupole mass filter 70, the quadrupole mass filter 70 is controlled by the controller to filter the precursor ions according to the m/z subrange being analysed. In some embodiments, each precursor m/z subrange may have a m/z range of at least: 20, 50, 100, 200, 300, 400 or 500. As such, the number of m/z subranges to be analysed will depend on the m/z range of each m/z subrange and the size of the m/z range of interest. For example, in some embodiments, the m/z range of interest may be split into at least 3, 4, 5, 7 or 10 m/z subranges. The benefit of having multiple TOF MS1 analyses of different m/z subranges is to maximise the dynamic range of each MS1 analysis. Accordingly, the plurality of MS1 analyses performed by the TOF mass analyser 150 may detect a relatively low-abundance precursor ion species which may not be detected by the MS1 analysis performed by the orbital trapping mass analyser 110 (e.g. due to the lower dynamic range of the orbital trapping mass analyser 110). That is to say, the TOF mass analyser may be operated to perform an MS1 analysis having a higher dynamic range than the MS1 analysis performed by the orbital trapping mass analyser.

According to this disclosure, the dynamic range of a mass analyser may be a ratio of the largest detectable signal to the smallest detectable signal of the mass analyser. One way of characterising dynamic range of a mass analyser may be a ratio of the intensity of a peak having 95% intensity of the mass analyser detection range to a peak having 5% intensity of the mass analyser detection range.

In some embodiments, the TOF MS1 analyses may have precursor m/z subranges of differing size. For example, in some embodiments, the mass range of interest may be split into a predetermined number of subranges based on a previous analysis. For example, a previous analysis may be a previous MS1 analysis or previous gain control analysis (discussed below). The previous analysis may indicate one or more precursor ion species having a relatively high abundance (e.g. the highest abundance precursor ion, or the top few e.g. 3 highest abundance precursor ions). Accordingly, the TOF MS1 analyses may subdivide the m/z range of interest such that the identified precursor ion species are excluded from some, or all, of the precursor m/z subranges. It will be appreciated that relatively high abundance precursor ion species will be identified in the MS1 analysis performed by the orbital trapping mass analyser 110. Thus the m/z subranges for the MS1 analyses performed by the TOF mass analyser 150 may be directed towards identifying lower abundance precursor ions. By excluding relatively high abundance precursor ion species from one or more m/z subrange, the dynamic range of the TOF mass analyser may be further directed towards detection of low abundance precursor ion species.

The (filtered m/z subrange) precursor ions pass from the quadrupole mass filter 70 through to the C-trap 100 as described above for the MS1 scan. The controller then controls the C-trap 100 to allow the precursor ions to pass through in an axial direction towards the fragmentation chamber 120 and on to the extraction trap 140. As the precursor ions are to be analysed in the MS1 domain, the precursor ions are not fragmented in the fragmentation chamber 120. The precursor ions are accumulated in the extraction trap 140 prior to analysis by the TOF mass analyser 150. The precursor ions may be accumulated in the extraction trap 140 for a predetermined time.

Precursor ions are then injected from the extraction trap 140 into the TOF mass analyser 150. The prior accumulation of the precursor ions in the extraction trap 140 allows the precursor ions to be injected as a packet into the TOF mass analyser 150. The packet of ions travels along the flight path of the TOF mass analyser before being detected at the detector 180. The varying arrival times of the precursor ions within the packet allows an MS1 mass spectrum for the packet of precursor ions to be generated. For the TOF of FIG. 1, the length of the flight path of the TOF mass analyser 150, in combination with the time resolution of the detector allows the TOF mass analyser to perform an MS1 analysis at a resolution of about 40,000 to 100,000, depending on the intensity of the analyte peak.

Each MS1 analysis performed by the TOF mass analyser 150 has a duration which is largely governed by the time taken to transport ions from the quadrupole to the extraction trap 140, and onward to the detector (i.e. the flight time of the ions). For example, in the embodiment of FIG. 2, each TOF MS1 analysis has a duration of about 5 ms. As such, it will be appreciated that each MS1 analysis performed by the second mass analyser (TOF mass analyser 150) may have a duration no greater than: 15%, preferably 10%, more preferably no greater than 5% of the duration of the MS1 scan performed by the first mass analyser (orbital trapping mass analyser 110).

As shown in FIG. 2, the TOF mass analyser 150 performs an MS1 analysis of each m/z subrange sequentially. Following the MS1 analyses, the TOF mass analyser then performs a plurality of MS2 analyses. The TOF mass analyser 150 may perform MS2 analyses for the remainder of the analysis cycle. The nature of the MS2 analyses to be performed will depend on the type of analysis being performed by the tandem mass spectrometer, which is discussed in more detail below.

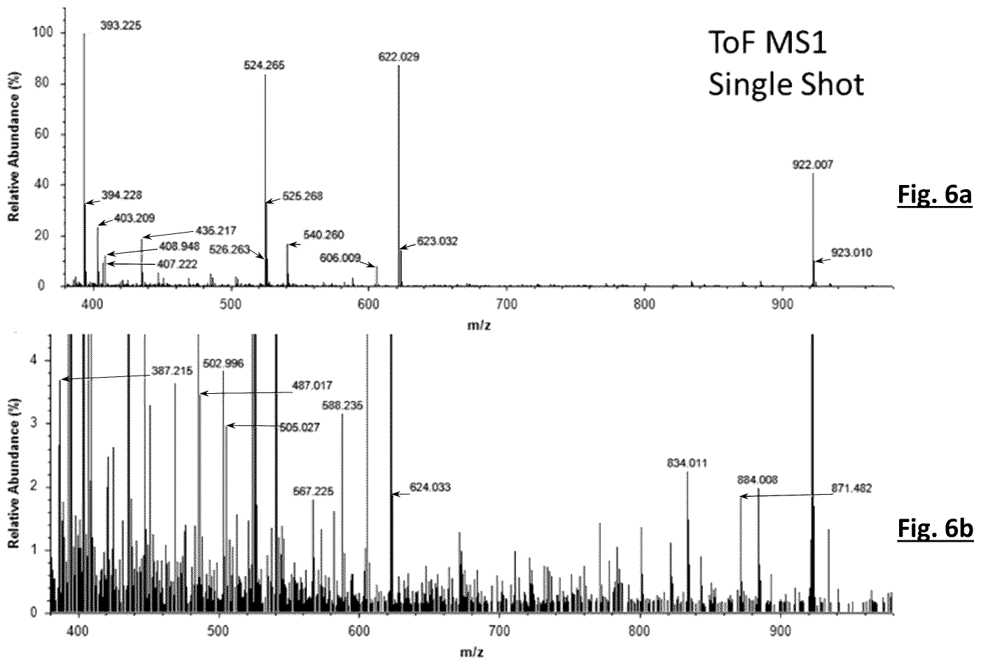

By way of example, FIGS. 6a and 6b show a graph of a mass spectrum obtained by the TOF mass analyser 150 following an MS1 analysis of the sample (calibration solution) across a m/z range of 380 to 980 m/z. FIG. 6a shows all the peaks recorded by the TOF mass analyser 150, while FIG. 6b shows a magnified view of the precursor ion peaks of FIG. 6a having a relative abundance of less than 5%. The mass spectrum of FIGS. 6a and 6b shows all the precursor ion peaks detected following a single MS1 analysis (a “single shot” analysis). It will be appreciated that, relative to the MS1 analyses performed by the orbital trapping mass analyser 110, the MS1 analysis of FIGS. 6a and 6b captures more low abundance analyte peaks than even the 240K resolution mass spectra of FIGS. 5a and 5b, due to the single ion level detection property of the TOF mass analyser 150.

It is possible that some of the precursor ion peaks recorded in the mass spectrum of FIGS. 6a and 6b are false positives resulting, for example, from random collisions of precursor ions with gas during acceleration into the analyser. One way of improving the mass spectrum is to average a plurality of mass spectra obtained using the TOF mass analyser 150 and apply a suitable threshold to remove noise. Thus, in some embodiments, the TOF mass analyser 150 may be configured to perform a plurality of MS1 analyses of the mass range of interest. That is to say, the TOF mass analyser 150 repeats the MS1 analysis of the mass range of interest a number of times. For example, the MS1 analysis may be performed at least 5, preferably at least 10, times. The data from the plurality of MS1 analyses performed by the TOF mass analysers may then be averaged together to produce an averaged MS1 spectrum from which precursor ion peaks may be detected.

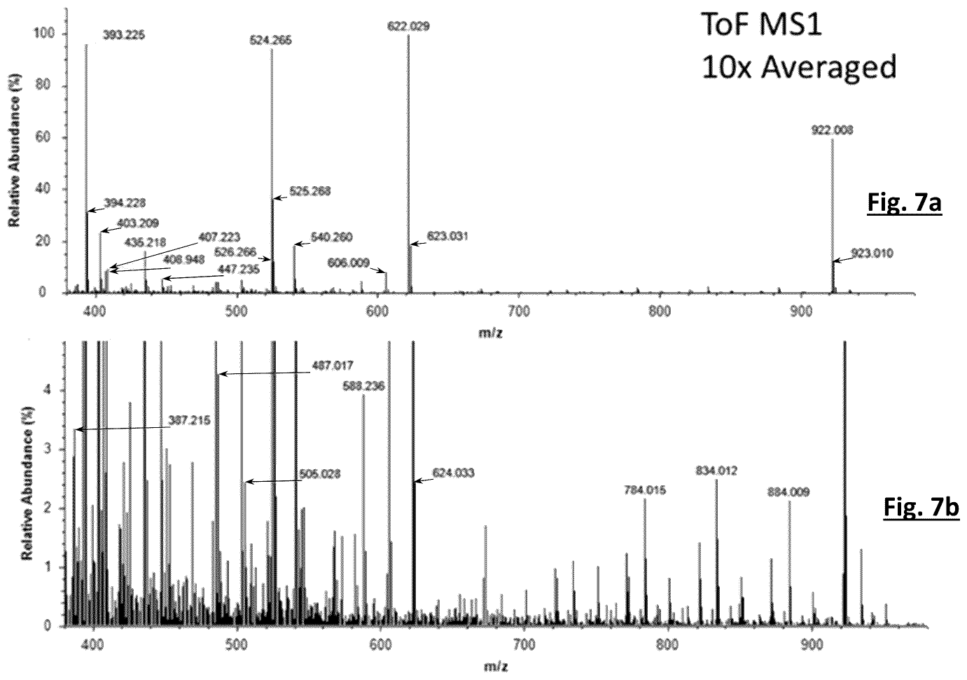

For example, FIGS. 7a and 7b shows show graphs of an averaged mass spectrum obtained by performing 10 MS1 analyses of the sample (calibration solution) using the TOF mass analyser 150 across a m/z range of 380 to 980 m/z. FIG. 7b shows a magnified view of the precursor ion peaks of FIG. 7a having a relative abundance of less than 5%. The same Pierce® FlexMix® sample is used to generate the precursor ions analysed in FIGS. 6a and 6b, and analysed in FIGS. 7a and 7b, in order to illustrate the effect of averaging the MS1 analyses. As will be appreciated from FIGS. 7a and 7b, the number of precursor ion peaks detected is increased relative to FIGS. 6a and 6b.

In addition to performing MS1 analyses using the TOF mass analyser 150, the TOF mass analyser may be used to perform MS2 analyses.

In order to perform an MS2 analysis with the TOF mass analyser 150, sample molecules from the LC column are ionised and injected into the tandem mass spectrometer 10 in a similar manner to the process for performing an MS1 analysis.

Precursor ions travel from the ion source 20 to the quadrupole mass filter 70. Precursor ions in the quadrupole mass filter 70 may be mass selected, wherein the mass selected precursor ions are subsequently transferred to the fragmentation chamber 120 for fragmentation. It will be appreciated that the precursor ions may be mass selected based on various criteria, depending on the type of analysis being performed.

For example, in some embodiments where a Data Dependent Analysis (DDA) is being performed, a combined list of precursor ion peaks (i.e. an inclusion list) may be generated based on the analyses of the precursor ions performed in the MS1 domain. For each precursor ion on the combined list of precursor ion peaks, the precursor ions may be filtered to select precursor ions having a corresponding m/z, and to exclude precursor ions having a different m/z. For example, the quadrupole mass filter 70 may be configured to mass select the precursor ions with a relatively narrow mass selection window of no greater than 5 amu, preferably 2 amu, more preferably 0.7 amu, centred on a m/z of a precursor ion peak of interest included in the combined list of precursor ion peaks.

The mass selected precursor ions are then transferred to the fragmentation chamber 120 where the mass selected precursor ions are fragmented to produce fragment ions associated with the precursor ion of interest. The fragment ions are then transferred to the extraction trap 140 and on to the TOF mass analyser 150 which determines the m/z of the fragment ions.

Each MS2 analysis performed by the TOF mass analyser 150 may have a duration which is similar to the duration of the MS1 scans performed by the TOF mass analyser 150. For example, in the embodiment of FIG. 2, each MS2 analysis performed by the TOF mass analyser 150 may have a duration of about 5 ms.

The tandem mass spectrometer 10 may perform a plurality of MS2 analyses using the TOF mass analyser in tandem with the MS1 analysis being performed by the orbital trapping mass analyser 110. This is discussed in more detail below.

In some embodiments, the tandem mass spectrometer 10 may also be configured to perform a gain control analysis (also known as Automatic Gain Control). The gain control analysis is configured to determine the precursor ion current. For example, the quantity of sample molecules eluting from the chromatographic apparatus may vary over time (e.g. due to a chromatographic peak), causing an associated fluctuation in precursor ion current. The gain control analysis may be performed to determine the precursor ion current at a given time, wherein the injection time for precursor ions into the C-trap 100 or the extraction trap 140 can be adjusted by the controller. For example, injection times can be adjusted to ensure a relatively consistent amount of ions are analysed in each MS1 analysis. The gain control analysis may be performed by the first mass analyser (orbital trapping mass analyser 110) or the second mass analyser (TOF mass analyser 150). As such, the gain control analysis may be a process for calibrating the number of precursor ions present in an MS1 analysis or an MS2 analysis.

As shown in the embodiment of FIG. 2, a gain control analysis (labelled AGC pre-scan) may be performed by the TOF mass analyser 150 concurrently with the MS1 analysis performed by the first mass analyser (orbital trapping mass analyser). Thus, the gain control analysis may be used to adjust the accumulation time in the extraction trap 140 for the TOF mass analyser 150 to ensure that a consistent number of precursor ions are analysed in each subsequent MS1 analysis performed by the TOF mass analyser 150. In some embodiments, an AGC pre-scan may also be performed prior to the performance of the MS2 analyses in order to ensure that a consistent number of ion species are analysed in each MS2 analysis.

In the embodiment of FIG. 2, the flow chart depicts a method in which the TOF gain control analysis is performed, followed by TOF MS1 analyses, followed by TOF MS2 analyses. Of course, in other embodiments, the analyses performed by the TOF mass analyser 150 may be performed in any order. For example, the TOF mass analyser 150 may be configured to perform a gain control analysis at any time during the methodology. That is to say, the TOF mass analyser 150 may, in some embodiments perform a gain control analysis clustered together with MS1 scans, but this need not be the case, for example they may be spread among the MS2 scans or triggered out of a regular sequence by observation of chromatographic peaks revealed by other scans such as AGC scans.

As discussed above, a packet of ions may be travelling through the TOF mass analyser 150 while the next packet of ions is being accumulated in the extraction trap 140. Thus, the TOF mass analyser may be used to perform a plurality of MS1 and/or MS2 analyses concurrently with the MS1 analysis performed by the orbital trapping mass analyser 110.

In the embodiment of FIG. 2, the TOF mass analyser 150 analyses the precursor ions across the m/z range of interest using a plurality of MS1 analyses. As shown in FIG. 2, the m/z range of interest is m/z 300-1100. For the MS1 analyses performed by the TOF mass analyser 150, the m/z range of interest is subdivided into a plurality of m/z subranges and an MS1 analysis is performed across each m/z subrange of interest. Subdividing the m/z range of interest allows the TOF mass analyser 150 to adjust the parameters of the TOF mass analyser 150 based on the m/z subrange in order to improve the sensitivity of the TOF mass analyser 150 for the m/z subrange being analysed. Thus, subdividing the m/z range of interest for the MS1 analyses performed by the TOF mass analyser 150 may improve the detection of low abundance precursor ions in the MS1 domain.

It will be appreciated that the m/z range of interest may be subdivided according to various criteria which may be specified by a user. For example, the m/z range of interest may be subdivided into at least 3, 4, 5, 7 or 9 m/z subranges. In some embodiments, the m/z range of interest may be divided equally between the m/z subranges of interest. In some embodiments, a user may specify one or more relatively narrow m/z subranges in order to target the detection of precursor ions having a specific m/z. In other embodiments, each m/z subrange may have a m/z range of about 50 m/z, 100 m/z, 150 m/z, 200 m/z or 300 m/z for example, wherein the number of m/z subranges is adjusted based on the m/z range of interest.

Each MS1 analysis performed by the TOF mass analyser 150 in the embodiment of FIG. 2 may have a duration of about 5 ms. Thus, it will be appreciated that a number of MS1 analyses may be performed by the TOF mass analyser 150 concurrently with the MS1 analysis performed by the orbital trapping mass analyser 110.

In addition to the method outlined with respect to FIG. 2 discussed above, a method 200 of tandem mass spectrometry is provided. FIG. 3 shows a flow chart of method 200 of tandem mass spectrometry in accordance with embodiments of the disclosure. It will be appreciated that the blocks in the flow chart of FIG. 3 correspond to the similarly named blocks shown in the block diagram of FIG. 2. As such, the discussion of the various steps of FIG. 2 provided above may also be applied to the steps of the method 200 discussed further below.

As shown in FIG. 3, the method 200 comprises performing a gain control analysis 201 (AGC Pre-Scan), performing an MS1 analysis using a first mass analyser 202 (Orbitrap MS1 Scan), and performing one or more MS1 analyses using a second mass analyser 203 (TOF MS1 Scan(s)). In the embodiment of FIG. 3, these steps are performed sequentially, while in other embodiments, some of the steps may be performed in tandem. For example, steps involving the first mass analyser may be performed in tandem with steps involving the second mass analyser.

Following the MS1 analyses, in step 204 (Build Unified Precursor List) the method 200 builds a combined list of precursor ion peaks. The combined list of precursor ion peaks may comprise a list of m/z of precursor ion peaks identified from the MS1 analyses performed by both the first and second mass analysers. In some embodiments, the combined list of precursor ion peaks may be filtered as part of step 204 to remove precursor ion peaks associated with singly-charged precursor ions. In some embodiments, the combined list of precursor ion peaks may be filtered to remove multiple charge states of a precursor ion from the combined list. As such, in some embodiments, the combined list of precursor ion peaks may be filtered such that only one charge state of a precursor ion is included in the combined list of precursor ion peaks. As discussed further below, where precursor ion peaks associated with a plurality of different charge state are present on the combined list, the precursor ion peak having the lowest mass to charge ratio (i.e. the highest charge state) may be maintain on the combined list. Alternatively, in some embodiments, the charge state of the precursor ion having the highest intensity precursor ion peak may be maintained on the combined list (with the other precursor ion peaks being filtered out).

In step 205 (TOF MS2 scan), the method 200 comprises performing an MS2 analysis using the second mass analyser. The MS2 analysis fragments and analyses a precursor ion having a mass to charge ratio corresponding to a mass to charge ratio of a precursor ion peak selected from the combined list of precursor ion peaks. The method 200 then repeats the MS2 analysis until each precursor ion corresponding to a precursor ion peak on the combined list has been analysed, or until a predetermined time limit has been reached. As such, the method 200 attempts to analyse each precursor ion in both the MS1 and MS2 domain within an analysis cycle.

As shown in FIG. 3, at step 206 the method decides whether to continue performing MS2 analyses, or to repeat the method cycle 200 again. As shown in FIG. 3, the method cycle 200 may be repeated once all the precursor ions have been analysed, or once a time limit is reached. As such, the analysis cycle may be repeated a plurality of times over the duration a sample elutes from a chromatographic apparatus.

In some embodiments, the maximum time allowed for each cycle of the method 200 may be specified by a user. For example, a maximum time allowed may be no greater than about: 1 s, 500 ms, 400 ms, 300 ms or 250 ms. In some embodiments, the maximum time allowed for each cycle of the method 200 may be based on the duration of the MS1 analysis performed by the first mass analyser (e.g. orbital trapping mass analyser 110). That is to say, the maximum time allowed for each cycle 200 may be set in order to ensure that the first mass analyser is utilised for the majority of the maximum cycle time. For example, in some embodiments, a duration of an MS1 analysis performed by the orbital trapping mass analyser 110 may be about 256 ms (at a resolution of 120,000), or about 512 ms (at a resolution of about 240,000) and a maximum cycle time may be about 270 ms or 530 ms respectively. Preferably, the maximum cycle time may be chosen to ensure that the first mass analyser is utilised for at least: 60%, 70%, 80% or 90% of the maximum cycle duration.

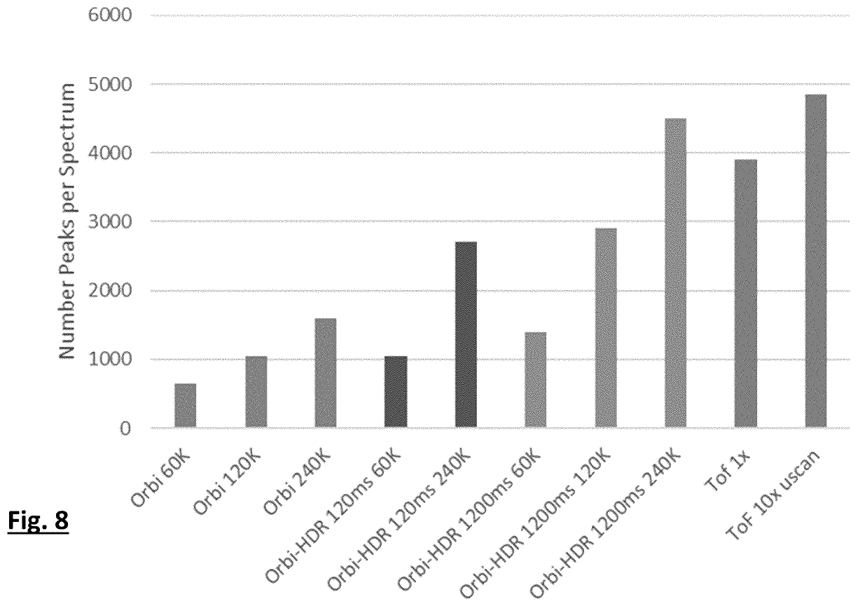

One important advantage of the method 200 is that the TOF mass analyser 150 is used to perform at least one MS1 analysis in order to identify precursor ion peaks for the combined list of precursor ions. By using the TOF mass analyser 150 in this manner, a substantial increase in the number of precursor ion peaks identified for analysis in the MS2 domain may be provided, as illustrated in FIG. 8. FIG. 8 is a bar chart indicating the number of different precursor ion peaks identified using MS1 analyses of a calibration solution (Pierce® FlexMix®). As shown in FIG. 8, MS1 analyses of a calibration solution were performed using the orbital trapping analyser 110 operated at resolutions of: 60,000 (Orbi 60K); 120,000 (Orbi 120K); and 240,000 (Orbi 240K). As shown in FIG. 8, when operating the orbital trapping mass analyser at a resolution of 240,000, fewer than 1,800 precursor ion peaks were identified. By contrast, a single MS1 scan using the TOF mass analyser of the same calibration solution identified about 3,900 precursor ion peaks.

For comparison, MS1 analyses were also performed using the orbital trapping mass analyser 110 operating in a “HDR” data acquisition mode. For the HDR data acquisition mode, the mass range of interest was divided into 12 windows (i.e. 12 mass subranges), wherein an injection time of up to 120 ms was allowed for a predetermined quantity of precursor within each mass subrange to accumulate in the C-trap 100 prior to injection into the orbital trapping mass analyser 110. As such, once the injection time for a given mass subrange reached 120 ms, the accumulation of ions for the mass subrange would end, even if the predetermined quantity of precursor ions was not reached. The accumulated ions were then mass analysed by the orbital trapping mass analyser 110. As shown in FIG. 8, HDR analyses of the calibration solution were performed using different resolutions of the orbital trapping mass analyser: 60,000 (Orbi-HDR 120 ms 60K); 120,000 (Orbi-HDR 120 ms 120K); and 240,000 (Orbi-HDR 120 ms 240K).

As a further comparison, the HDR data acquisition mode was also performed using an injection time of 1200 ms. While such a long injection time is impractical for normal mass spectrometry use, the relatively long injection time ensured that for each mass subrange the predetermined quantity of ions was accumulated in the C-trap 100 prior to mass analysis with the orbital trapping mass analyser. As shown in FIG. 8, HDR analyses of the calibration solution were performed using different resolutions of the orbital trapping mass analyser: 60,000 (Orbi-HDR 1200 ms 60K); 120,000 (Orbi-HDR 1200 ms 120K); and 240,000 (Orbi-HDR 1200 ms 240K).

FIG. 8 also shows data for the number of precursor ion peaks detected by the TOF mass analyser when performing MS1 analyses of the calibration solution. FIG. 8 shows data for the number of precursor ion peaks detected from a single scan, and from an averaged mass spectrum obtained by performing 10 MS1 analyses of the calibration solution. As shown in FIG. 8, a single scan performed by the TOF mass analyser 150 detects more precursor ion peaks of the calibration solution than every MS1 analysis using the orbital trapping mass analyser 110 barring the HDR analysis performed at 240K with 1200 ms injection time. For reference, a single MS1 analysis using the TOF mass analyser can be performed in about 5 ms, which is significantly shorter than the 1200 ms injection time used for each of the 12 mass subranges forming part of the HDR analysis. Furthermore, the time-averaged mass spectrum obtained using the TOF mass analyser 150 identified more precursor ion peaks than any other analysis and may also be performed on a timescale which is compatible with a normal mass spectrometry workflow (e.g. as shown in FIG. 2). Thus, it will be appreciated from FIG. 8 that using a TOF mass analyser 150 to identify precursor ion peaks may provide an effective method of identifying precursor ion peaks which would not otherwise be detected using an orbital trapping mass analyser 110.

In some embodiments, when generating the combined list of precursor ion peaks (e.g. step 206 of FIG. 3), the second set of precursor ion peaks may be thresholded. That is to say, the intensity of each precursor ion peak in the second set of precursor ion peaks may be compared to a first predetermined intensity level (threshold). Any precursor ion peaks below the first predetermined intensity level may be removed from the second set of precursor ion peaks (i.e. not used to generate the combined list of precursor ion peaks). By removing relatively low intensity precursor ion peaks from the second set of precursor ion peaks, e.g. single ion signals (i.e. peaks representative of single precursor ions which have been scattered during analysis) may be removed from the second set of precursor ions. Thus, the second set of precursor ions peaks is more representative of the precursor ions present in the sample (calibration solution) by reducing the effect of precursor ion scattering during mass analysis.

In some embodiments, the second set of precursor ion peaks may also be thresholded to remove any precursor ion peaks above a second predetermined intensity level (threshold), wherein the second intensity level is greater than the first intensity level. As such, precursor ion peaks having a relatively large intensity, for example having a signal strength representative of at least 10 precursor ions, may be removed from the second set of precursor ion peaks. Precursor ion peaks of relatively large intensity are likely to have been detected by the first mass analyser (orbital trapping mass analyser 110), and so can be removed from the second set of precursor ion peaks to reduce repetition of precursor ion peaks. That is to say, the second intensity level (threshold) may be set at a level corresponding to a precursor ion peak intensity which will be detected by the first mass analyser as a precursor ion peak of the first set of precursor ion peaks.

In the embodiment of FIG. 1, the TOF mass analyser 150 outputs a voltage signal representative of the precursor ions arriving at the detector. As such, the first and second intensity levels may correspond to voltage levels which are used to detect the presence of precursor ion peaks. In some embodiments, the controller may comprise a digitiser configured to apply the thresholding discussed above.

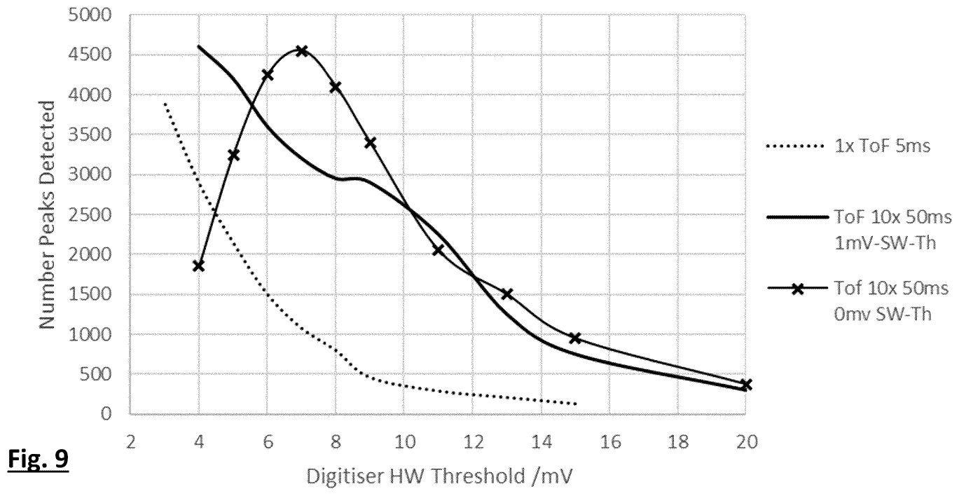

FIG. 9 shows a graph indicating the number of precursor ion peaks detected using the TOF mass analyser as the first intensity level is varied. In the embodiment of FIG. 9, the TOF mass analyser 150 was calibrated, and noise level checked so that false peaks arising from electronic noise were negligible at all threshold levels shown, thus all detected peaks related to ion signal, rather than electronic noise. In the embodiment of FIG. 1 and as shown in FIG. 9, the first intensity level was varied from about 1 mV to 20 mV. In other embodiments, the first intensity level may depend on the nature of the signal output from the TOF mass analyser but may generally be configurable to either allow single ion precursor ions peaks to be detected, or to exclude precursor ion peaks representative of one or two precursor ions.

As shown in FIG. 9, the number of precursor ion peaks detected by a single scan using the TOF mass analyser 150 (1× ToF 5 ms in FIG. 9) decreases as the first intensity level (Digitiser HW Threshold) is increased from 1 mV to 20 mV. This is expected as increasing the first intensity level causes an increasing number of single or well-separated multiple ion peaks to fall short of the threshold. In particular, multiple reflection TOF mass analysers such as TOF mass analyser 150 often have a detector response which may be smaller than the peak width. As such, the small precursor ion peaks detected may form a plurality of spaced-out single ion events, which have similar height (though far greater peak area) than a single ion event comprising a plurality of precursor ions.

FIG. 9 also shows a graph indicating the variation in number of precursor ion peaks detected using the TOF mass analyser 150 when a time-averaged acquisition method is used for different first intensity levels (TOF 10× 50 ms 0 mV SW-Th). By using a time-averaged acquisition method, a more complete picture can be obtained regarding which of the relatively low intensity precursor ion peaks are reproducible. It will be appreciated that the time averaged graph indicates that more precursor ion peaks are detected than for a single TOF mass analysis for first intensity levels above 4 mV. This indicates that much of the detected peaks in the TOF mass analysis scans relate to “real” ion events (i.e. precursor ions of different mass to charge ratios), rather than as a result of the scattering of precursor ions.

It will be appreciated that for the time-averaged graph (TOF 50 ms 0 mV SW-Th), at lower first intensity thresholds (e.g. from 6 mV to 1 mV) the number of precursor ion peaks starts to decrease. This decrease is due to the presence of scattered ion signals (i.e. false ion signals) appearing in the time-averaged data. The increase in such peaks causes the spectra to become so dense that scattered peaks in close proximity become merged, making them difficult to distinguish as individual peaks.

To address this problem, the time-averaged data may be further thresholded to remove single ion peaks (which are likely to be representative of scattered ions). Thus, by thresholding the time averaged data with a third intensity level, lower than the first intensity level threshold (e.g. 1 mV in FIG. 9), the single ion peaks may be removed prior to the detection of the precursor ions. Thus, as shown in FIG. 9, when a time-averaged graph is generated and a further 1 mV threshold applied (TOF 50 ms 1 mV SW-Th), the number of precursor ion peaks detected increases as the first intensity level is reduced, mirroring the behaviour of the single TOF mass analysis graph.

As will be appreciated from FIG. 9 and the above discussion, the above-described methods may generate a combined list of precursor ion peaks comprising a substantial number of peaks (e.g. FIG. 9 indicates that up to 4500 precursor ion peaks may be detected by TOF mass analyser 150 when analysing the calibration solution). Of these precursor ion peaks, a significant portion will have a relatively low signal intensity. Such relatively low intensity peaks may be difficult to assign a charge state to (e.g. the precursor ion peak may be associated with a singly charged precursor ion, or a multiply charged precursor ion). In general, knowledge of the charge state of the precursor ion which gave rise to a given precursor ion peak may be useful for setting fragmentation energy for the subsequent MS2 analysis of the precursor ion. Furthermore, singly charged precursor ions may be less useful for MS2 analysis than their multiply-charged counterparts. Thus, in some embodiments, the combined list of precursor ion peaks may be filtered to remove singly charged precursor ions. The assignment of charge states to precursor ion peaks may be performed by a suitable software algorithm. Known algorithms for determining charge state include Thorough High Resolution Analysis of Spectra by Horn (THRASH) (D. Horn et. al, Journal of the American Society for Mass Spectrometry, vol. 11, issue 4, April 2000), and Advance Peak Detection (APD) algorithm, available from Thermo Fisher Scientific®.

Thus, in some embodiments the controller may be configured to determine a charge state for each precursor ion peak on the combined list of precursor ion peaks. The controller may determine the charge state associated with each precursor ion peak using a charge state determination algorithm. In some embodiments, it may be desirable to analyse each precursor ion in the MS2 domain only once. As such, where the same precursor ion appears on the combined list of precursor ion peaks under different charge states, the controller may filter the combined list of precursor ion peaks such that only one charge state of each precursor ion appears on the combined list of precursor ion peaks. Preferably, the controller may filter the list to remove precursor ion peaks associated with multiple charge states, wherein the precursor ion peak associated with the highest charge state for each precursor ion is maintained (i.e. the precursor ion peak having the lowest m/z for each precursor ion). Alternatively, in some embodiments, the charge state of each precursor ion having the highest intensity precursor ion peak may be maintained on the combined list of precursor ion peaks (with the other precursor ion peaks being filtered out). In this way, the combined list of precursor ion peaks for analysis in the MS2 domain may be optimised to reduce, or prevent, repeated MS2 analysis of the same precursor ion.

It will be appreciated that the assignment of charge states to each of the precursor ion peaks in the combined list of precursor ion peaks may require some computational time. In some embodiments, the tandem mass spectrometer 10 may be provided with an ion mobility separator (not shown in FIG. 1) which is configured to filter singly-charged precursor ions from the precursor ions provided to the orbital trapping mass analyser 110 and the TOF mass analyser 150. For example, ions of different ion mobility may be filtered by the ion mobility separator according to a compensation voltage applied to the ion mobility separator. One known ion mobility separator is the FAIMS Pro® Interface available from Thermo Fisher Scientific®.

It will be appreciated that the MS1 analyses performed by the second mass analyser (TOF mass analyser 150) may be performed at any time during an analysis cycle. For example, in the embodiment of FIG. 2, the MS1 analyses performed by the TOF mass analyser 150 are performed towards the start of each MS1 analysis performed by the orbital trapping mass analyser 110. For example, the MS1 analyses performed by the second mass analyser 150 may be performed during an initial period of the MS1 analysis performed by the first mass analyser (orbital trapping mass analyser 110). In some embodiments, the initial period may be no greater than: 30%, or 20%, of the total (expected) duration of the MS1 analysis performed by the first mass analyser. As such, it will be appreciated that the initial period extends from the start of the MS1 analysis performed by the first mass analyser to the end of the initial period (i.e. when 30% of the total (expected) duration of the MS1 analysis has passed).

In some embodiments, it may be advantageous to distribute the MS1 analyses performed by the second mass analyser (TOF mass analyser 150) throughout the duration of the of the MS1 analysis performed by the first mass analyser (orbital trapping mass analyser 110). As such, in some embodiments where a plurality of MS1 analyses are performed by the second mass analyser, the MS1 analyses may be distributed evenly across the duration of the MS1 analysis performed by the first mass analyser. That is to say, the plurality of MS1 analyses to be performed by the second mass analyser may be interleaved with the MS2 analyses to be performed by the second mass analyser.

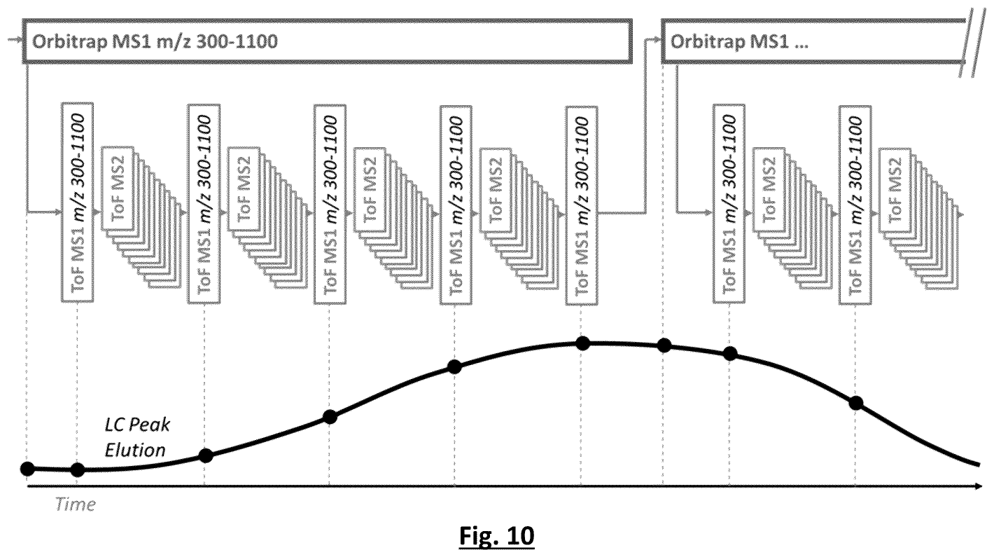

For example, FIG. 10 shows a block diagram of an analysis cycle performed by the tandem mass spectrometer 10 of FIG. 1. In the block diagram of FIG. 10, the MS1 analysis performed by the orbital trapping mass analyser 110 may have a duration of about 256 ms. FIG. 10 shows for comparison a profile of a chromatographic peak eluting from the chromatographic separation apparatus over the duration of the analysis cycle. It will be appreciated that the MS1 analysis performed by the orbital trapping ions takes a sample of ions from the beginning of the elution of the chromatographic peak. As such, for relatively fast eluting chromatographic peaks, it is possible that the MS1 analyses performed by the orbital trapping mass analyser may not fully characterise the chromatographic peak. For example, in FIG. 10 only two or three MS1 analyses may be performed by the orbital trapping mass analyser 110 over the duration of the relatively fast eluting chromatographic peak.

To improve the characterisation of the chromatographic peak, in FIG. 10 a plurality of MS1 analyses are performed by the TOF mass analyser 150 concurrently with the MS1 analysis performed by the orbital trapping mass analyser 110. In particular, the plurality of MS1 analyses performed by the TOF mass analyser 150 are interleaved with the plurality of MS2 analyses performed by the TOF mass analyser. In the embodiment of FIG. 10, at least 5 MS1 analyses are performed over the duration of a single MS1 analyses performed by the orbital trapping mass analyser 110.

In the embodiment of FIG. 10, the plurality of MS1 analyses may be interleaved with the MS2 analyses by performing an MS1 analysis followed by a predefined number of MS2 analyses. For example, an MS1 analysis may be repeated after every 5, 10, 15, or 20 MS2 analyses performed by the TOF mass analyser 150. As such, the analyses performed in the MS1 domain may be interleaved evenly throughout the duration of the analysis performed in the MS1 domain by the first mass analyser. Alternatively, the MS1 analyses may be performed at predetermined time intervals during the MS1 analysis performed by the orbital trapping mass analyser 110.

It will be appreciated that in the embodiment of FIG. 10, each of the MS1 analyses performed by the TOF mass analyser 150 is performed across the entire m/z range of interest. In other embodiments, MS1 analyses may be performed across m/z subranges of interest (e.g. as described above in relation to FIG. 2), where the analyses are interleaved throughout the duration of the MS1 analysis performed by the orbital trapping mass analyser. In some embodiments, each MS1 analysis of a m/z subrange of interest may be repeated a plurality of times throughout the duration of the MS1 analysis performed by the orbital trapping mass analyser 110.

In some embodiments, the data from the MS1 analyses performed by the first and second mass analysers may be used to identify a chromatographic peak eluting from the chromatographic separation apparatus. For example, the data from the MS1 analyses performed by the TOF mass analyser 150 may be used to identify and/or characterise the chromatographic peak shown in FIG. 10. In some embodiments, the analyses to be performed by in the MS2 domain by the TOF mass analyser 150 may be selected based on the identified chromatographic peak eluting from the mass spectrometer. For example, the combined list of precursor ion peaks may be updated based on the chromatographic peak identified. In particular, the order of precursor ions to be analysed in the MS2 domain on the combined list of precursor ion peaks may be updated based on the identified chromatographic peak.

Thus, in accordance with the above description, a method of tandem mass spectrometry, a tandem mass spectrometer, a computer program and a computer-readable medium are provided.

1. A method of tandem mass spectrometry for analysing precursor ions across a mass to charge (m/z) range of interest comprising:
analysing some of the precursor ions across the m/z range of interest in the MS1 domain using a first mass analyser of a tandem mass spectrometer operated at a first sensitivity;
analysing some of the precursor ions across the m/z range of interest in the MS1 domain using a second mass analyser of the tandem mass spectrometer operated at a second sensitivity, wherein the second sensitivity is higher than the first sensitivity,
wherein the analysis in the MS1 domain performed by the second mass analyser is performed concurrently with the analysis performed in the MS1 domain by the first mass analyser;
combining data from the MS1 analyses performed by the first and second mass analysers to identify and/or quantify precursor ions; and
analysing some of the precursor ions in the MS2 domain using the second mass analyser of the tandem mass spectrometer.

2. A method according to claim 1, wherein
the first mass analyser is operated at a first sensitivity and a first mass accuracy; and
the second mass analyser analyses some of the precursor ions in the MS1 domain at a second sensitivity and a second mass accuracy, wherein the second mass accuracy is lower than the first mass accuracy.

3. A method according to claim 1, wherein
combining data from the MS1 analyses performed by the first and second mass analysers comprises generating a combined list of precursor ions peaks in the MS1 domain.

4. A method according to claim 3, wherein
the combined list of precursor ion peaks comprises a first set of precursor ion peaks identified from the MS1 analysis performed by the first mass analyser and a second set of precursor ion peaks identified from the MS1 analysis performed by the second mass analyser, wherein the combined list is filtered to remove precursor ion peaks which are repeated between the first and second sets of precursor ion peaks.

5. A method according to claim 4, wherein
the second set of precursor ion peaks is thresholded to remove any precursor ion peaks below a first predetermined intensity level and/or any precursor ion peaks above a second predetermined intensity level.

6. A method according to claim 4, wherein
a first precursor ion peak generated by the second mass analyser and a corresponding first precursor ion peak generated by the first mass analyser are used to calibrate the tandem mass spectrometer, wherein the calibration is used to identify precursor ion peaks which are repeated between the first and second sets of precursor ion peaks.

7. A method according to claim 1, wherein
the analyses performed in the MS2 domain are based on the precursor ions identified by the MS1 analyses.

8. A method according to claim 1, wherein
analysing the precursor ions in the MS2 domain comprises:
fragmenting the precursor ions to generate product ions; and
analysing the product ions using the second mass analyser.

9. A method according to claim 1, wherein
the method comprises performing a plurality analysis cycles using the tandem mass spectrometer, wherein each cycle comprises:
performing a single analysis across the m/z range of interest in the MS1 domain using the first mass analyser; and
performing a single analysis across the m/z range of interest in the MS1 domain using the second mass analyser; and
performing analyses of some of the precursor ions in the MS2 domain using the second mass analyser,
wherein the analyses performed in the MS1 and MS2 domains by the second mass analyser are performed concurrently with the single analysis in the MS1 domain performed by the first mass analyser.

10. A method according to claim 1, wherein
the method comprises performing a plurality analysis cycles using the tandem mass spectrometer, wherein each cycle comprises:
performing a single analysis across the m/z range of interest in the MS1 domain using the first mass analyser; and
subdividing the m/z range of interest into a plurality of m/z subranges and performing an analysis across each m/z subrange of interest in the MS1 domain using the second mass analyser; and
performing analyses of some of the precursor ions in the MS2 domain using the second mass analyser,
wherein the analyses performed in the MS1 and MS2 domains by the second mass analyser are performed concurrently with the single analysis in the MS1 domain performed by the first mass analyser.

11. A method according to claim 10, wherein
each cycle comprises: performing a plurality of analyses in the MS1 domain for each m/z subrange of interest using the second mass analyser, and
for each m/z subrange of interest averaging the data from the MS1 analyses performed by the second mass analyser.

12. A method according to claim 1, wherein
the method comprises performing a plurality analysis cycles using the tandem mass spectrometer, wherein each cycle comprises:
performing a single analysis across the m/z range of interest in the MS1 domain using the first mass analyser; and
performing a plurality of analyses across the m/z range of interest in the MS1 domain using the second mass analyser; and
performing analyses of some of the precursor ions in the MS2 domain using the second mass analyser.

13. A method according to claim 12, further comprising:
for each analysis cycle:
averaging the data from the analyses performed in the MS1 domain by the second mass analyser.

14. A method according to claim 12, wherein
averaging the data from the analyses performed in the MS1 domain comprises thresholding the data to remove precursor ion peaks below a third intensity level.

15. A method according to claim 12, wherein
the plurality of analyses performed in the MS1 domain by the second mass analyser are interleaved with the analyses performed in the MS2 domain by the second mass analyser.

16. A method according to claim 15, wherein
for each analysis cycle, at least 3, 5, or 7 analyses are performed in the MS1 domain by the second mass analyser.

17. A method according to claim 15, wherein
the analyses performed in the MS1 domain are interleaved evenly throughout the duration of the analysis performed in the MS1 domain by the first mass analyser.

18. A method according to claim 8, wherein
each analysis cycle further comprises performing a gain control analysis using the first mass analyser or the second mass analyser, wherein an injection time for each of the MS1 analyses performed by the first and/or second mass analysers is adjusted based on the gain control analysis.

19. A method according to claim 1, wherein
the precursor ions to be analysed are filtered to remove or reduce the number of singly-charged precursor ions.

20. A method according to claim 1, wherein
the precursor ions to be analysed are provided by an ion source which is configured to ionise molecules provided from a chromatographic separation apparatus.

21. A method according to claim 20, wherein
data from the MS1 analyses performed by the first and second mass analysers is used to identify a chromatographic peak eluting from the chromatographic separation apparatus.

22. A method according to claim 21, wherein
the analyses to be performed by in the MS2 domain by the second mass analyser are selected based on the identified chromatographic peak eluting from the mass spectrometer.

23. A method according to claim 1, wherein
the first mass analyser is a mass analyser selected from the group comprising: an orbital trapping mass analyser, a Fourier-transform ion cyclotron resonance (FTICR) mass analyser, and a Time Of Flight (TOF) mass analyser; and/or
the second mass analyser is a TOF mass analyser.

24. A method according to claim 1, wherein
the first mass analyser is operated at a first dynamic range to perform the respective MS1 analysis; and
the second mass analyser is operated at a second dynamic range larger than the first dynamic range to perform the respective MS1 analysis.

25. A tandem mass spectrometer for analysing precursor ions across a mass to charge (m/z) range of interest comprising:
a first mass analyser configured to analyse precursor ions in the MS1 domain; a
a second mass analyser configured to analyser precursor ions in the MS1 domain and the MS2 domain; and
a controller configured to:
cause the first mass analyser to analyse some of the precursor ions across the m/z range of interest in the MS1 domain at a first sensitivity;
cause the second mass analyser to analyse some of the precursor ions across the m/z range of interest in the MS1 domain at a second sensitivity, wherein the second sensitivity is higher than the first sensitivity,
wherein the second mass analyser analyses the precursor ions in the MS1 domain concurrently with the first mass analyser analysing the precursor ions in the MS1 domain;
combine data from the MS1 analyses performed by the first and second mass analysers to identify and/or quantify precursor ions; and
cause the second mass analyser to analyse some of the precursor ions in the MS2 domain.
